SEQUENCE LISTING (14 sequences)

SEQ ID NO: 1 - DNA, 6802 bp, Homo sapiens
cagcagggtcacaagggcaggccgggtccttgtggagagcacatttagtgggagggacatgatttcccttcaaagtgcccattctggacgcttcccgttccatgctggacgcttcctcttccacgctggatgcttcctgttccacactggatgcttcctgttccacgctggatgtttcctgttacactctggatgcttcctgttccacactggatgcttcctgttccatcctggatgcttcctgttccatgctggacatttcctgttccactctggatgctccctgttccatgctggatgcttcctgttccatgctggatgcttcctgttccatgctggacatttcctgttccactctgcatgcttcctgttccactctggatgcttcctgttccatggtggacgtttcctgttccactctgcatgcttcctgttccatgctggatgcttccttttccattctggatgcttcctgttccatgttggatgtttcttgttccactctggatgcttcctattccattctggatgcttcctgttccatgctggacatttcctgttccatgctggatgctttctgttacatcctggatgcttcctgttccatgctggatgttttttgtttgactctggatgcttccagttccattctggatgcttcctgttccatgctggatgcttccttttccattccgcacaattcctattccattctggacacttcctgtgcgacacctccttgggttttctgtctgcccagtccctctatcctcatcccgttccctgctacctcccacctccacaatcgtccttgcccagctcctccctctctctagagcttcggcctggcaaggtccctcctgatctcagtccaggctcccccagcacaggtaggagcctagcacctgcccttggacctccccaccctgcatggtgccagcatcccccggtccccagggaggccccatttctctctctgcttgtagtccagtggccctggagtcccactgcaactcgggtgtgcccctgacctctgaggaagttaagtgtcctgtccctagccaggctatcccgtctgctcagccccagggccctgcccccaaccccttcccctcacctgcaccacaggctctggccaactctgcccaggccctgaatgggcccctctggctcccctctgctgctacactgccctgcaccacctccactcagcttcagtgtgttcatccacctgtcccaagtcccctcggcccccaggagcacagctggtggccctggctcctggcagcccatcttgttccttctggagcaccagcctcagaggccttcctgtgcagggtccacttggccagccctgggaccctcctggtctcaagcacacacgttctccctgcagccagacctgcccctgcctgtgagctcagccctgagccttggaatgccttcccatctccatcccagctcgcctttgccagctgctcagcaggatgaactcacactcccctccctgcaccatgagtcagagccagctggagacacgcccaggccaaagcagccaccagggcctagtgggggccagaagcttcagatgagaggcccaggtattgagaggctgagatcacgggcagaatggtcataatcgctgccagtatcagtccagccccagggactcagagacagagaaaagagcagcacacaaggtctgggctccccaccttctcccgtgagtacgggggagtatgggggcagccaccacccccatccccacacacccatgaggcagcctcggctgtgtctggactccccctcgccctctgacccagaaaccaccagaagaaaagggaacttcaggaagtaagtggtgccgccggtttcaatcctgttcttagtctttgcagcgtggagttcacacacctggggacctgggggccgagctgtgatttcctaggaagacaaatagcggctgacggcaggggcggggctgcccacatgtacctcgccagaacaggaagggctgagacccccacctcggtgagtggggtcagcacagggcaggggcacaggctcgggaggaggacagagcctgggggcagccgtgggcgctcctggacctgagatgctgaacaggctccaagaggctggggagacatggggtcgaggccggccccacatggaggcccaagcggagccagcacgggggaggtgggcagccttcaggcaccgatgcccacccagtgcgagacgacggggaccgtgggcaggggcttccaagccaacagggcaggacacaccagaggctgactgaggcctccaggacgaccgggctgggagcacgaggaacatgactggatgcggcagagccggccgtggggtgatgccaggatgggcacgaccgacctgagctcaggaggcagcagagcgagggaggaggagaggccccaggtgaacggaggggcttgtccaggccggcagcatcaccggagcccagggcagggtcagcagtgctggccgtggggccctcctctcagccaggaccaaggacagcaggtgagccgggagcagagcagggagggtgagtgtggcagcaggacaggagggtggaagccaaggagcccagaggcagaggcagggacaggggaggcacaggggctgggctcagagccagctgatggggttggggcacctgctggcggggagcagggctgtggtcagcagtggagaggaggggagagctgtgctgagtgcacgggcgggagaagggaagagtccagggaggcccagaaaggcccagagtgcagcaggcctggggcgaggggaagggctgaggctccgtgcgttcagggaactgacccagcagagcagaggccactgaggagctgaggttccagagaggcttccagagcaggagcagtgcagggacaggaggatccgggagctcattcaggaggggcacatgggcaagggcaaggggctctgttggggagacctgactggacactggggctgctccacagcatagggaacacgccaagtgctgcaaaatcaaaaatgagggcagaaaaacagcccaaacctggacagagggtgccaggacaggcaggggggcaacagtgacctgagtgacattgctgcccgggttgagggagggcagagtgagcagggagcaggcattggagctcagggaccaggaccaagcagccacaggtgagcagggcaggtgggggcagaaggagcagggggcacctcctggagctcaggggaccagggcagagcagcctcaggtgagcaggggctggtgggcggcaggatgagcagggggaagaccctggagctcaggggaccagggcagagcatcagaaggtgagcatggctagtgggagatgggcaagcagggggcagcccctggagctcaggggaccaggacagagcatcaggaggtgagcatggctagtgggaggtgggcgagcagggggcagcccctggaactcaggggaccagggcagagcagccgcaggtgagcacgggctggtgggaggcgggaggaacagggggcagctcctggacttcaggggaccagggagggcatctgaaggtgaacaggggccagtggggggcaggatgagcagggggaagctcctggagctcagggagccaaggcagagcagccgcaggtcagcaggggcaggtgggaagcatggggagcaggtgggcagcccctggagctcagagagccagggcagatcatccacaggagagcaggggctggtaggaagcaggaggagcaagtgggcagcttttggagctcagagcaccagggcagaagagcctcaggtgagaaggggcaggtgggaggcagaataagcaggggacagcccctggacctcaggagaccagggcagagcatcacaacgtcagcatggctggtgggaggtgggcgagcagggggcagcccctggacctcagagagccagggcagatctgcaggtgagcaggggcaggtgggaggcaggaagagcaggaggcagctcctggagctcaggggatcagggcagagcagccacaggtgagcaggggcaggtaggaagcagaaagatcaggggtcagcccctggagctcaggggacaaggggagagcatcagaaggtgagcaggactgaggctcagcctcagggagccagggcagagcagctgcaggtgagcagggccggtgggaagcaggaggagcaggtgggcagcccctggagctcagagagccagggaagatcatccgcaggtgagcaggggctggtgggaagcaggaggagcaaggggcagctcctggagctcaggggaccagggcagagcagtcgcaggtgaacaggggcaggtggggggcaggaggagcaaggagcagctcctggagctcaggggaccagggcagagcagtcgcaggtgaacaggggcaggaggagcaaggggcagctcctggagctcaggggaccagggcagagcagccgcaggtgagcaggtgcaggtggggggcaggaggagcagggggcagctcctggagctcaggggaccagggcagagcagccgcaggtgagcaggggcaggtggggtgcaggaggagcagggggcaggcactggagctcaggggaccagggcagagcagtcgcaggtgaacaggggcaggtggggggcaggagtagcaaggggcagctcctggagctcaggggaccagggcagagcagtcgcaggtgaacaggggcaggtggggggcaggaggagcagggggcagctcctggagctcaggggaccagggcagagcagccgcaggtgagcaggtgcaggtggggggcaggaggagcaggggtcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtcagcagggccggtgggaggcaggacgagcaggggacaggcactagagctcagggcaaggcagccacaggtgagcagggctggtgggaggcatcactcagctcctagactttggcaggagctgggtagttgctggcaacagacagctgagggctggtgaaagtgcagtgcagcctcctggtgccgggaagggagtgtgagtccatcccactgagcagttggcaagggcgagctgggatggagaagggaaggcgttccagggctcagggctgagctctcaggcaggggcaggtgtggctgcagggggaacgtgtgcttgagaccaggagggtcccacggctggtcccagcggaccctgggcaggaaggcctctgaggctggcgccccagaaggagcaagatgggctgccaggagccagggccaccagcacaatgaagctgagtggaggtggtgcagggcagtgtagcagcagagggcagccagaggggcccattcagggcctgggcagagtcagccagagcctgtggtgcaggtgaggggaaggggtggtgagcggggccctggggctgagcagaggggatggcctggctgagggcagggcgcttagcctcctcagaggtcaggggcacaccccacctgcagtgggactccagggccactgggccagcggcagagagaaatggggcctccctgtggcctgggggtcctggcaccatgcagggtggggagggccaagggcaggtgcaaggctcctacctgtgctggggggcctgggttgagcccagcagggaccttgccgggggaagctctggagagagggaggaggtgggctggtggccgagaaggccaggccagggctgggagggtgacggtgtggtgactgagcctccagaagtaatgcaggacactgggaggcagggggcatccaggcactcagggccctgacctgggctgctgcacactggggctaaggggaaaggaggggagaggctgaggaggaggctccaggaggctattccaaggcagggggttccggggccctggggctgaagggcgccgaccctatgcagtgtctggcccctctgctgcacagaagaaaagggccttggagggcagagggcaggctatgaccagggccctgggcaagtcaggccaactcactaggggagggccacgctggggcggcagggtcaggggcttcagggggctcgggggacccacgagaagccatctgagaacagtgtccactggtcaagccaggcacccataaaaggctggagtggggccaatgggcatgagccgtccctgaggtggcaccgatggccagagctgaggccaagctagaggccctggactgtgctgactcccggcagacacagagcgctgacctggctgccgagccccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcag

SEQ ID NO: 2 - DNA, 9483 bp, Bos taurus
gatcaaacactgtcaaaccaacacaagagagccccagtaccagatgtgaggctccccccagaccccagtcacagtgcagcctgtagagggaacaggcccagtcgggagaccaggaccgagaaagtgacccctgtcagtcagtccagctactcagtcatgtccgactctttgcaaccccacagactgcagcacgccaggcctccctgcccatcaccaactcccggagcttgctcaaactcaagtccattgagtcggcgatgccatccagccatctcatcctctgtcgtccccttctcctcctgcccccaatccctcccagcatcagagtcttttccaatgagtcaactcttcgcatgaggtggccaaagtattggagtttcagcttcaacgtcagtccttccaatgaacacccaggactgatctcctttacaatggactgattggatctcattgtagtccaagggattctcaagagtcttttccaacagcacagttcaaaagcattgattttttactcagctttctttatggtccaactctcacatccatacatgactactggaaaaaccatagctttgactagacggacctttgtcagcaaagtaatatctctgctttctaatgtgctgtctacgttggtcatagcttttcttccaaggaacaagtgtcttttaatttcatggctgcagtcaccatctggagtgattttggagcccaagaaaatagtctgtcactgtttccattgtttcctcgtctattttgccatgaatgccatgatcttcatttttcgaatgttgagttttaagccagctttttcactctcctctttcactttcatcaagagtctctctagttcctcttcattttctgccatgagagtggtgtcatctgcatatctgaggttattgctatttctcctggtaatcttgattccagcttgtgcttcctccatccaggtatgtcacatgatgtactctgcatagaagttaaataagcagggtcacaaaatacaatcttgatgttctccttttccaaattttaaccagtccgttgttccttatctggttctgttacttcttgacctgcatataggtttctcaggaggcagttaatgtggtctggtattcccctctctttaagaatgttccagtctgttgtgatccacccattcaaaggctttagtgtagtcaataaagcagaagcagtaaaatgctgccactttctaggtggagctcacatatggataccacacccaaaccttgaccaagccagaacctcatgacaatcatgtgcagtgctataaatgatctcagctgttacacagtcactatttacaaaatgtggggcaagagagcctgaacatgccatcttggccaattttctctctcctcatgggttgtttagctttttttctttttttttttttttaattaagctgtatgagttgcttgcacattttggaagtttattctttagtggtcacgtgatttgcaaatatcttctctaattctgcaggtagtcttgttttgttgatggttttccatggtgtacaaaagccttttagcttaattaggtcccatttgtttatttttctttttatttctattactctaggagatggatggagaaggaaatggcagcccactccagtgttcttgcctggagaatcccagggacgggggagcctggtgggctgccgtctatggggttgcactgagtcggacatgactgaagtgatatagcattagcattaggagataaatacaaaaagacaccaccttgatttatgtcaaagagtgttctgcccatgtttccctctgttttatagcctcccatttcacacttttgtctttaatccattttgagtttatttttgcccatggcactagagaatttataatttcatttattaacaggtagcaatccagtttttccagaacttattcaagagactgtcttttctccatcttacactcttgcctcctgggtcatacatgaattgatcataggtgggtgggattgttcctgggctgtctatcctgtctcactgatcactgatctataattgtggttttgtgccagcaccatagtgttttgatggccgtagctttgcagtatgctctgaagtctgggagcctgatgcctccagctccacttttgtttcttgagattgctttgactatttggggtctttatgtctcccaacaaattttcaaaagttttgttctgggtctgtggaaattgccattggtcatttgataggtgttacactgaacctgtagattgctttcggtagaagtgtcattggcacaatattgattcttctgatttaagagtatcttttctaaatagacatttctccaaaaaagacatacagatggctaacaaacacatgaaaagatgctcaacatcactcattatcagagaaatgcaaatcaaaaccactatgaggtaccatttcacaccagtcagaatggctgcgatccaaaagtctacaagcaataaatgctggagagggtgtggagaaaagggaaccctcttacactgttggtgggaatgcaaactagtacagccactatggagaacagtgtggagattccttaaaaaactggaaatagaactgccttatgatccagcaaccccactgctgggcatacacactgaggaaaccagaagggaaagagacacgtgtaccccaatgttcatcgcagcactgtttataatagccaagacatggaagcaacctagatgtccatcagcagatgaatggataagaaagctgtggtacatatacacaatggagtattactcagccattaaaaagaatacatttgaatcagttctaatgaggtggatgaaactggagcctattatacagagtgaagtaagccagaaggaaaaacataaatacagtatactaacgcatatatatggaatttagaaagatggtaacaataacccggtgtacgagacagcaaaagagacactgatgtatagaacagtcttatggactctgtgggagagggagagggtgggaagatttgggagaatggcaatgaaacatgtaaaatatcatgtaggaaacgagttgccagtccaggttcgatgcacgatgctggatgcttggggctggtgcactgggacggcccagagggatggtatggggagggaggagggaggagggttcgggatggggaacacatgtatacctgtggtggattcattttgatatttggcaaaactaatacaattatgtaaagtttaaaaataaaataaaattggaagaataaaaaaaaaaaaagagtatcttttcatctgtttgtgtcaccttaatttctttcatcagtgtcttagagctttcagagcagaggaattttgcctcctagggaaggtttattcctaggtattttattctttttgatgggatggtaagtgggattctttccttaaattctctttatggtatttcattgctgccatacagaatgcaacagatttttgtgtattaattttgtatccataaattttatccaattcattcctgagctctagggttttctgtaccttctttagcattttgcatgtgttaaatcatgtcacctgcaaacagcaggtgtttcttacttggcttctggattccctgtgtccctttctctcccctgattcttacacttaggccttcactgtgctgcactgtgggcccttgctggttacctactatatacgcagtcgtgtggatgtattagcaccaaactcctaatttatccctgggatgtggtgggtcaaccaggcgcagtgcatgaggaagaggaacaccgtgacggctaggattcccactcctcccctgggtgcaagggcgccgcttaaaccaccccaccaacccacaagacatacctcagtcacatctggactcccccaggccctctgagacagaaaacactcagaagaaaagggaacttcaggaagcaagtcgcgtcaccaggtttcattcctgttcttagtcttcacagcactgggggaagggccctcacacctcctgggactggtgaccaagtcccagggagcagggctgcagacacagaacacagggaacttcaggaggcagatggagtcaccaggtttcattcctgttcttagtcttcacagcactgggggaagggccctcacgcctcctgggactggtgaccaagtcttagggagcagggctgcagaaacagcacgcagatcccaggagccacatgtgtctgtgggccagagcagggaaggggcccggagccccaggctgtggggcagccccctccatggcccccaccatgggtgagtggggaccccgaggatgagcgcacagggacaggaatgagagccctggagggagaccggcctcgggacgcccagaggggctggagcagccgagtgggcccaggggaggcagctgcccctggtcacagtgcaggacgagcgtgtggacagaacccagacagggtgcaccacaccaagagcactgctgcccagggctggagagctcagggggctgtgggccgtggaggagcaggatgggcttgggggcgggagggaagggatgtgcatgaccaggggctctggaggccggggctgggcagtcatgaggaccagccaggacggaaagagggtctgacctgaggacgcagggaagggagaggaccccagagcagtgaagtcttctggacaggccagtggagccagcgtcggtcccagggcagggccacatgcagggactgtgggtcccgactgggctggggccgagggcagcggcagacttgggggcaaggctgcagggccagccgggttctatgtccacagcaggcaccacggggctgaacccacagccccccaggacacagggggctgaccagagctcagggcctgaggaccagcagtgggagccagagagaggcaggtgacccgaaaactggaggcagcaaggggccagaggagggtgtggatggagggatagagaagagcagagagcaaaggcaggacctctgccaggaggggcccgggtggacagagctcctccaggggagagttgcctggaggccagtgggcacctggcaagtccagggatgagagctgaacccaggaaataaaggagtgaggagtggagatgggcaggggtgagggcttccagaggggccagaggggcaagaaaggctgagggggcggcccaaggacagggggcagcaaggctgcaggtgagcaggggctgggagggcaggggggcccctagagatctgggggagcaggcacagcttgtgggtgaaaaggggttaggagggcagggggcccctggtgtttgggggagcagatagagcttttgtaggtgagcagggagctgggagggcgggggagagggtggcctggtgctttggggagcaggtacagcctgtgagggtgagcaggggctgggagggcagggggtgttcctggtgtttggggtagaaggcacagcttatgggtgagcaggggctgggagggcagggggagcccccggtgctttgggggagcaggtactggttatgagggtgagcaagggctgggaggacggggggagcccccagtgctttgggggaacaggtacaggttatgagggttgagcagggggctgagagtaaggaggtgagcctagtgctttgggggagcatgtacagcctgtgaaggtgagcaagggctaggaggacagtgggatcccctggtgctttaggggagcaggtacagcttgtgagggtgagcaagggactgggaggacaaggggtgcccctggtgtttgggggagcaggtactagttatgagggtgagcaggggctgggaggactgggggagcccctagtgctttgggggagcaggtacaggttatgagggtgagcaggggctgggaggacagggggtgttcctggtgtttggggtagaaggcacagcttatgggtgagcaggggctgggaggacaggggggcccctggagatatggggaagcaggtacagcttgttggtgagcaggggctgggaggacagggggagcccctggtgctttgggggaacaggtacaggttatgagggtgagcagggggctgagagtaaggaggtgagcctagtgctttgggggagcatgtacagcctgtgaaggtgagcaagggctaggaggacagtgggatcccctggtgctttgggggagcaggtacaggttatgagggtgagcaaggggctgggaggacaaggggtgcccctggtgctttgggggaacaagtacagcctgtgacggtgagcagggagctgggagggcagggggtgcctctggtgctttgggggagcaggtacagcttgtgagtgtgagcaagggctgggagaactgggggtgtccctggtatttgggggagcaggtactggttatgagggtgagcaggggctgggaggactgggggagcccccagtgctttgggggagcaggtacaagttatgagggtgagcagggggcttagagtaagggggggtgcacctggtgctttggggagcgttacagcctctgagggtgagcatgggctgggaggacagtgggatcccctggtgctttgggggagcaggtacagcctgtgagggtgagcaggggctgggaggacagggggagcccttggtgtttgggggagcgtgtacagcctgtgagggtgagcaggggctgggaggaaagtgggagcccctggtgtttgggggagcaggtacagcctgtgagggtgagcaggggctgggaggaaagtgggagcccctggtgtttgggggagcgtgtacagcctgtgagggtgagcaggggctgggaggaaagtaggagcccctggtgtttgggggagcgtgtacagcctgtgagggtgagcacgggctgggagggcagggggagcccctggtgctttgggggagcaggtacaggttatgagggtgagcaaggggctgggagggcagggggagcccctggtgtttgggggagcatgtacagcctgtgagggtgagcaggggctgggagggcagggggcgcccctggtgttttggggagcggtacagcttgagagggtgaacaggggctgggaggacagggggagcccctgatgctttgggggagcaggtacagcttgtgagtgtgagcaagggctgggaaaactgggggtgtccctggtgtttgggggagcaggtactggttatgagggtgagcaggggctgggaggactgggggagcccgtggtgctttgggggagcaggtacaggttatgagggtgagcagggggctgagagaaaggggggtgtgcctggtgctttggggagcattacaatctgtgagggtgagcaggggctgggagggcagggggagcccctggtgctttgggagaacaggtacagcctgtgagggtgagcaggggctgggaggacagggggagcccctggtgtttgggggagtgtgtacagcctgtgagggtgagcaggagctgggagggcagggggagcccctggtgctttgggggagcaggtacaggttatgagggtgagcaggggctgggaggactgggggtgtgcctgatgctttgggggagcaggtacagcttgtgagggtgagcagggggccgggaaagcagggtgtatctcaaaaactttaggggatcaggtagtgcttctggggatgagcagaggtctgggaggacagggggtgcccttggagttggagagcaggtacagtctgtgaggtgagtagagggctgggcaggcacagggagcccctggtgctttgggggaacaggtgcaggtcgtgagggtgagcaggggctgggaggacagggagtgtccctggtgtttggggtagaaggcacagcttatgggtgagcagggcctgggaggacagggggagcccctgttgctttgggggagcaggtacagcttgtgagggtgagctgggtctgggaagacagggggagcccctggtgctttgggggagcaggtacagcttgtgagggtgagctgggtctgggaagacagggggagcccctggtgtttgggagagcagatacagcctgtgagggttagcaggggctgggaggacagggggtgcccttggagttggagagcaggtacagtctgtgaggtgagtagagggctgggtgggcagggggagcccctggtgctttgggggaacaggtaaaggttatgagggtgagcagggggctgagagtaagggcgtgtgcctgttgctttggggaagcatgtacagcctgtgagggtgagcaggagctgggagggcaggggggagcccctggtgctttgggggagcaggtacagcctgtgagggtatccagggggctgggaggacatggggtgcccctggagatttttggaagcagtaatagcttgtgggtgagcaggaagctgcgaggacaggaggtgcccctgttgctttgggtgaacaggtacaacttgggtgagcaggggctgggaggacagggagtgcccttggagatttgggggagcaggtgcagcttctggggataagcagggggctgggagttcagggtattaggggagcagacaaagatcgcgcagctgagcaggcgctggaatgtcacgcccctgccgacgacctcagttgaccatgtgtgtgctgagcacatcggtacgaaagggtccgagggtgcaaggggccatttgtgctgtgggccagatgcagggacacagggcagtgtgagccctgcagagaagatggggcccaagagcgcagctgtccagagctgagtccagagggctgagaccagtggtgtgggtgctggacgagggtgaagggctggcgtgagggagcaagggcacagggctgggagagctcaggccacagctggtgaagcagagggctgagacaaggggtgtaggccacccctcaagacaatgggggtgctgtttggtggagggtctgcatgaagaacaaagcaaggaggagcgaagacccgagaggaagggctctggggctgcagggaaggggcgccctgagggcaccgggtgggctgcacttctgggccgagggtctgcctgtcccagcagcctgtgtggctcaggagccaaagtggggccagacctgggacgctgtgcttgatagggcggtgcaaggggccaggagctacagggcgcccactggcagtcagtcgggctgcaggcccagggagctgaccaggctgggggagcaggggccatgggaagggccaggcgcccacaggagggaaggggccctgggctctgatccaggaggccacactcaccgacccaagctgtgtccagacaccccaactgcagtacagagagccgggtggccaccatgccggccggtcatcagaccctggaagcaggtggtggctgggctcggaggtgccccaggcctgggcacctgaggtcctgctggaccccgcattcacccagcctcctctctcacag

SEQ ID NO: 3 - DNA, 10298 bp, Equus ferus
  misc_feature (6918)..(8037): n is any nucleotide
agatatcccccaagtcatgtcacaatactaccatgttcaaagtacatgaatgctttgaaacagactggtctcagtggctgggtgttccttgggtggctcctaatggaactcagtggttgtgtggaacaaacctctagtcttggctcctgcctggatggctgggacactgcaccctggacttcctttggaggcagggaaggagtcgccctgagctaacagctggtctcaatgttcctcccagtaaggccagatggactccatcagtgttcactggtatgaccaccagcggctatatttgctgcttccttgggctacaagatgtcatatcacatagaggctctaactaaatttactcaaactgccctcaatgaaagtcaggtaggaatacccttattaaatactcaaatgtctttagtggggagggctgtctccccatcagaatggccttagatagtctaacagcatcccaaggtgggacatgtgcactcattcaaactgaacgctgcgtttttagacctgatgagtcttccaatgtgtcctctctgctaaagcacttggaaaagcaggtaaatgccttaagcgatcctgcatccaggcttgatttgcgtggttggctcccttcaggtgtggctgccctcttgaaatctgaattgcaattcctgtgtctgttactccttggaattcttctattaatcataatatgcaaactaattactctttcctttactcagtgttgtaagactggcatgcaggctagaataatggttgctcagccacttgaggtgattgattgatcttacaaccctggatgaatttccttctcataagactatgcctaagaactcatttttcaaataatcctttcaagttttgaattatcagcactgttagtgtagctgttctataattaggcacatgcagcatcatgagtcagatcaaagcataggtacaacgtttgtgtgacaatctaaagttactgaaaacttacataacctatggaatgcttcccctgtaattgtatacctctatgctcatccactatatccaactatttcctgccattgggcataactgagcacgtcaatgagataaaagcccagcactgatggccatcatggaccagggcaggcaacaatcaccatagcaccaagggacaatatttagatcatcagtgctttctataaaaacattatcgatcaaaggtgggaaaataatgaatcaatgaaagcaataagaaaagttaaaatgttgaggtatatgaggaagccatttggtttagactaattaggcctcatctagtttttctggaaagtcctgatgtgcctgtcgaatatgcattgtacatctgctgcatttacaatgtcccaaagcaagaatgatgcccttgaaattatcctgcccccttttggcatttctttatagatgagcacttcttcccagaatgcaaggattcgttactgacctactgtgtcatcttgtgatcactcacccgcaatactagcaaagcatcttgtgactgtagtaaaagagatactcctgtcatatgtgatgtacgtcccttgttccaagacggtatataaccacgctgcacacccggcttcttcacaacacttccttccttggtgaaggttattgtcccgggctatgtagtcctcaaattggctcaaataataaactcaccccaattttgattaacagattgattatagtttattgcctcaacatttcctgaccagaaattacatagatgaacgtgcttcttatgtaacggcagcgatgctcacagcctgcggagcacatgactgagggaggctgccttccgtgggagagatggcaaaactgcgtgggcgccatggagaggaggccgggccagagccgccgggagtgtgcagtggattatcatgaattcatcccggagggaccagagggcactcccttcaccaaggctgctgctgacggcgttttctgggtgtccacagcaacattcccaggaccacaggctcctccatcacctggtcctgccccatgcagaggttcacttccctcaccagaccccagggtacccatgcgactgactccatggggacagggccactgagggaccactgctgggtctctgagagcaggacctaaggagacagggctcctcctgggagtcaggcccgggcaccactcagcgtgggcaatccccatggaaagatcacagagacagagggaacacgggcgcccgcccagcccaggcgccagactccaggtggagccttccagcccctcgctgatgcgagtggagcagagaggagcgcccccgcccacagaagccggcactgcctgcaggccaccaacgcagaagcaaatcagcagaagagctgcagaggccactctctgtggggcggtccacagcacacccatgaaccgccggacacgatatgggacccgcacgtgggcgctgctctgagaaacctcaactctagaaagtaagcacgaggaccagagagcaggggaggcagagggacccagagtagaccgttgggggatgtggagtggctctggggacacccgaggccaaagggtgctgcaaatagcaacaggtgggagggccgactcccctcgtacatctcaggtcacctcctagaaggttcgctttggactttgctcttggcccacctaagcggccacagcgtacgcccctgcccaccaaccattggaaggagccaggcctgccttcacgtccagtcacctacctggcccccggcaattccagggtggggaccatggtgccgcccagggatccccgatccaaggcctaacgtgcaagagacgggatggcccaggtttgtacacggggccttgggtgtgccgaggcactgcaagtcatgagacactgagggtccgtgtccccccacagacaaaaagctgggtcctggcccacacacgggaggacaggaacagcatgctgtctctcagacagaggagacctttgggccccagtgaccaccgtggactctgtccttgtacatagacttctttctgtccccagaggacagctgtggatgggagaatatcttccctgggaccctgggtgctaccacccttcagtcagagatccagccatggatggagccagagagggatggagggaagaggcagggacccagaggaagacggctttgtacttagggggctggcctggcaggaggacaggatgaggccctgggctgagctgcgggctgtgagcaggacagcctgtgtccacaatggacgctgactagggcagggggaggtgtcctctgggctgtgggtaccagagggctcaggtgagaggcctgggtgccctggcctttggtggggccatcgggcataatccttgtgggagggagagagcaaagagaggcatgtgagcccggacttcctgcactctccccagagtcctgggcgccagaccccctccactcccacacacccacgtgtggcagcctctgcctcacgtctgtactccccccggctctctaagacagaaaccacccagaagaaaagggaacttcagggagcaagcggtgcccctgctttcagtcccgttcttagtctttgcagggtcgtggagagtgggttcccacctccgggaaccagctaccacctccgggaaccagctaccaactcctaggaagcagggcagagggcaacaagcccggggccgcgaggagccacatgtgcccggggccagagcaggggcgtggggggagctcctcggaagccctggcttccaccacctccacagcccccacccttggtgagtggggtccccacggagcagggggaagggagtggagtcagggcagtggaaacggttgcagagctacggggagcccaggcacccagggacgactccgaggggctcaagcagctgagccagcacgggggagggcaggctgcccacgagccacggtgccaaccggacgcaggcaccaggcagccatgggcagggcacgcccacgccaacagagctggacacaccagaggcccatgaaagttaaccttgaggaaattgatgaccttgaggccagaagcctgagggctgcatttggccagctgtgctgagggcaggaccagccaggagctgagctgctgcagcacggacgccaggatgcacagatgggtttgagcacagagagggggctggctcagagcaggggaggaagaggacctccaggtggcctaagggtcctgtccacgccaatgagccagctcaagccccagggcagggtcagaggtggccgccatggtgtcctaggctgtagccaggaccgagggcagcaggtgagccaggtgcagagcccaaagggccggcggatgcacccatgtgctcagaactcagactgcacagggctgggcaggagctcagtggctacaggcagacgttgggagccaggcagtgagggtccagagcaggtgctggggttgcagtgttgggagctgggtggcaaggggagctgacccagcaatggagggagcacagtcccgaggggccttccagatgggcttctgagcgccgtgaggggcacagagacggtgaagcccagctgggccaggagctcctccaggaggagaccacggcaggcaggggctctgccaggggagagttagccagagagagggtgggcatctggtggctccaggaggcaaaggccagtgatgggaaaggaatgagcccaggatggagggtgaacctgggctgagggtgctgggagaggcaggaggggcaaaggcagccccgggcagacccagcagcctggggctgagcttagggacctgagctgtgtggaggggatcgtaggacaggagggacagagggctgctgtggacctggagttccagggacaacgggaagaacagctacggatgagcaggggaaggtgggagggcacaaaaggacaggagcctacaccaagagatgcatgggggcaggcagagcagctacaggtgagcagaggccagtgggagggctggagggaaagggggctgcccaggggctctggaagggggcaggtagagcagctacaggtgagcagaggaatgggagaccaggagggacaggtcacagcccaggagccccatgggtgtggagagcagagcttggcatgtaagtaggaagcaggaagggctgaaggggcaggggctacccagcagctccacaggagcaggcagagaagctacaggtgagcaggggccggtgggagggctgcaggggcagggggctgcccaggagctccggggagcaggcagagcagctacaggtgagcaggggccgatgggagggctgcaggggcagggggctgcccaggagctccggggagcaggcagagcagctacaggtgagcaggggccgatgggagggctgcagggacctgcccaggagctctggcggagtagtcagagcagctacaggtgagcaggggccagtgggagggctggaggggcagggtcctgcccaggagttccaggggagcaggcagagcagctataggtgagcagacgctggtgagaggtctggaggggcagggggctgcccaggagctctagcgggagcaggcagagaagctataggtgagcaggggccggtgggagggctgcaggggccggggcctgccgaggagctccggcggggtagtcagggcagctataggtgagcaggggccggtgggagggctggaggggcagtgggatacccaggagctccgccagggtagtcagagcagctacaggtgagcaggggcaagcaggagggctggaggggcaggaggctgcccaggagctccggcggggtagtcagagcagctacaggtgagcaggtgccagcgggagggctggaggggcagggggctgcccaggagctctgggaggagcaggctgagaagctgtagttgagcaggggccagtgggagggctggaggggcaggcggctgcccaggagctccgggggagcaagcagagcagctataggtgagcaggggcaggtgggggggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggagcaagtgggagggctggaggggcagaggactgctcaggagctccgggggagcaggcagagcagctacaggtgagcaggagcaagtgggagggctggaggggcagaggactgctcaggagctccaggggagcaggcagaacagctactggtgagcaggggtcagcgggagcgctggaggggcagggggctgcccaggagctccgggggagcaggcagagcagctataggtgagcaggggcaggtgggagggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggggccggtgggagggctgcaggggcagggtcctgcccaggagctccacaggagcagacagagaagctataggtaagcagggccggtgggagagctggaggggcagggtccggcccaggagttccgggggagcaggcagagcaggtacaggtgagcaggcgctggtgagaggtctggagcggcaggtggctgcccatgagctctggggggagcaggcagagcagctataggttagcagtggccagcaggagggctggaggggcagggagcagttcaaggagctccagcggaataatcagagcagctataggtgagcatgggccagcgggagggactgggacggggcagggagctgccaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttccagggaatcttctacagggagctagatagagaaagtttataggtaaagcaggtgccggtcgggagaagttgaaggggcagggtcctgccccaggaagttcccggggggagccaggcagagcaggttacaggtgagcaggcgattgtgagaagtctggaagcggcaggtgtcttcccatgagctcttggggggagcaggcagagcagctataggttagcagtggccagcaggaggggctggaggggcagggggcagccaaggagctccagcggggtagtcagagcagctataggtgagcaggggccagcgggagggctggaggggcaggggacttccaaggagctccatgggtgcagacagagcagaaactggtgagcaggggccggtgagagatctggagttgcagggggctgcccaggagctccgggggagcaggcagagcagctactggttgcaggggcaggtgggagggctggaggggcaggggcctgcccaggatctccagggagcaggcagagcagctataggtgagcaggggcaggtgagagggctggaggggcagcgggctgcccaggagctccaggggagcaggcagagcagctactggttgcagggcaggtgggagagctggaggggcagcgtcctgcccaggagctccacaggagcagacagagaagctataggtaagcaggggccggtgggagagctgggggggcagggtcctgcccaggagttccgggggagcaggcagagcaggtacaggtgagcaggtgctggtgagaggtctggagaggcagggggctgcccacgactttaggggatgaagggcactgggcctgagttaaacagagccctcaactggtgggacctgagggggtacagggagcagggacagctggaggctcccagggctcaggtcaggctggctgggcagggaggagagttgagctggttgagttgccaggtgcctgttgagttggatggaggggggcccctgggatggaaggagtctagggctgaaggggtcgggggcatccctgagaggaccagatcggatgcagttcttgggtccaggcacaccggccctgttctggggtttgtggggacatgggacaggagggatagacattcggactcagtgtggggtggcacagcgcagtgggctcagtgcatggaagagtaagagtccccttggggcctgagcccaggctaccagcatggggagaggccaggcaaggccctgcggctgagcagacaggactcaggggaggaggggacagggtgggcctggctgttgggggtggaggctgcaccctggcccacacgggggcctggggaggctggcaccgagaggagaggagtgccgagcgcagcgcatggctcccgcccgcgcccaggggccggctgtgctggggaaggaccacagctggaggagggctctgcagtgagcaaggagctggacaggcccaagagcagggagggcagagggcaggcggcgctggcagggaggggccggaggtgagctcaggaagccgggccaggaggacctgagcgggtagggggcccagaggcacatgcaggaggctgggctggggacacacagggatgggaggggagggggacaagaagactcccagggatggggtccagtacacggatggagtccagggaagggacggggtccaggacagggaagggatgggttcaggacagggatggggtcgagggcaaggacaaagcccaaggcatgaacagggttcaagacaggatcaggatcaggggagcgttggggacaagtactcctggtgctgtggctgtggagctgactcagatgacacgtggcctggcgagcagagacaggccatggcaaagtcactgagctgaggggccgggagacaacagggacagtgcggcgcagaggcccaccacacctgaagagatgcccaatcgcccagggctgggccaggctggggccacaggacagcagattagggtccatcagaagccgaccagaagcacaggtctctggtccgggctcagactggggactcagtcaggcgggccacctgcaggaggcctgagcagggacaaggggcaggggccccagggggctctaatctaggttctctgagcctgtggctgcagaggcaccagccctgatgacaggcagccacaaggggcatctaggcctcagactctctggaaacacacgggcgcaggggcaggccctggtggtcacacgcctcctctcttgcag

SEQ ID NO: 4 - DNA, 9313 bp, Sus scrofa
ggtatatatacacactagaataccactcggccgtaaaaacgaaccaaataatgccattggcagcaacacggatggaaccagagactctcatactaagtgaagtaagtcagaaagaccaataccatacgctatcacgtacatctggaatctaatatatggcacaaataaacctttccacagaaaagaaacccatggacttggagaaaatacttgtggtcgccaagggtgaggtagtggggtagactgggagtttggggttagtagatgcaacatattgcatttggcatagataaactatgagatcctgctggagagcacagggagctgtatctagtcccttgtgatggaacatgttggaggataaagtgagaaaaagaacatataaatatatacaccacacgcacacacacacacatatatgaatgactgggtcactttgccgtagagtagaaattgacagaacacagtaaatcaactataacggaaaaaaataaaaaacattaaaaaaaaaaaaacagatgctcatctccatggattctgaacacatttttgacagaattcaacacccaactttttcttgatgatttttattttttccactgcaggtggtttaccctgctctgttgattccactgtacagaaaagtgacaaagtcacacacatatatatatgtagacattctttttctcacattatcctccatcatgccccatcataggtgactagatagagggaaactacaccaacatactaaaagccatatatgacaaacccacaactatcatcattctcaatggtgaaaacctgaaagcatttccgctaagatcaggaacaagacaaggatgtctgctctcaccactctccttccacggagttttggaagtcctagccagggcaattagagaagaaaaggaatccaaatcggaaacggagaagtaacactaccgctctttacaggtgacgtgctgcgatacctagagaatcctcaaggcactaccagaaacctcttagagctcgccaatgaatttggtcaagctgcaggatacaaaattaacacacagaaattggctgccttgctctacgttaacaatgaaagagcagcgagagaaactagggaaaccatcccactgagcagtgcctcgaaatgaatgaaatgcccaggaataaacctacccaaagacacaaagacctggactctgaaaaccataaggcactgctggaagcaaccaaagacgacacaaaccggtggaaagctagaccatgctcctgccctggaagaattaatactggcaaaagggccataccacctaaggccaccctcactctcagtgcaatccctctcaaatctccaatggcattttccacagaactagaacaaaaaacctcaaaaatttgcatggaaacacaaatacctcactgacctgggctatactacaaagctgcagtcatcaaaacggtatggcactgacactcccccccaaaaaagacatacatatcagtggaacagaacagaaagcccagcaataaacccaagcacctatgctcaatccatccatgacataggaggcaagactattcaacagagaagggagagtcacttcactaagtggtgctggggagctggacagctccatgtaaagaatgaaaccagaacactcccaaacgccaaacacaccaaaaaaaaacaaaaaacaaaaaaaaaaaaataaaaaaaccctcaaaacaaatgaaactcctaaacgtaagactggatacctataactcacagaggaaaacaggcagaacgttctttgacataaatcacagcaacatcttatttgatccacctcctggaataactacaataaagacaaaagtggagttcccgtcacggctcagtggtaaccaaatctgactaggaaccatgaggttgcgggttcaatccctggccttgatcagtgggttaaggatccggcgttgccgtcagctgtggtgtaggttgcagacgcggctcggatcccacgttgctgtggctctggtgtaggccgacagctacagctccaattcgacccctagcctgggaacctccacatgccatgggtgcggccctggaaaagacaaaaagacggggaaaaaatgaaagaaagaaagaaggaaagaaggaaaggaggaaagaagaaagaaagaaagaaagaaagaaagaaagaaagaaagaaagacagacagacaaaagtaaaccaatgagatctaattaaactcaaaagcttttgaaaagcaaaagaaaccattttaaaagagaaaaacaggagttcccgtcctggctcaggggttaacgaatctgactagcatccatgaggaggcagatttgatccctggcctcactcagcaggttaaggatccggcattgccatgagctgtgagctgtggtgtaggccagcagccgtagctccaattcaacccctagcccgggaacctccatatgccacaggtggggccctaaaaaccaaaaaagacaaaggaaaacaacccacagaatgggacaaaatctttgcaaacaatgcagcccaccaaggctcaatccccaaaatatacaaacactctacaactcaacaacaacaaaaaacaaaccaacaaacaacccaatcgataaatgggcagaagacctaaatagacatttctccaaagaagacatacagatggccagaagacacatgaaaaaatcctcaacagcactaatttttagggaaatgcaaatcaaaactgcaatgaggcaccacctcacactggtcagaatggccatcattaagagtcaactaacagcaaatgctggagagggtgaggagaagagggaacccgccttcactgttggtgggaatgcaacttggtacaagcactgtggaaaacagtatggaggaacctcagaaaactgaacgtaaaaccacgattgaacccagaaatctcacccctaggcctgtatctcgatacgactttcagtcaaaaacttacatgcacaactatggactttgcagaagggttcaaaatgggcaggacatgaaaacactcaaacgcccatcaacagacgaacggattaagatgtggttctcacacaaagtaagaactacgtctcagccgtaaaaaaggaccaagtcacgccatttgcagcaacgtggatggaactagagactctcacactgagtgaagtaagtcagaaagagaaagacagagaccacatgataccacttatatgtggaatctaaaatacggcgcgattgatcctgtggacaaaacagagacagagcatggtcatggagagcagactttgctttccagggggagggagagaagagattgacagggggtttggagctggtagatgcaactgatcacattgaaaacgagtaagtgatgggtcctactgggcagcacacggaacagtgcccggcctcttgcgttagaacgtgagagaggaaggagagtgtgaaaagaaaaagaaggtaaatgtatgtacagctgtacaagggtaattgaaggaacgttgtaaatcaactctggttttaaaaaaaaaaaaaaaaatttaaagaaagaaaacgttctccagaggcagaacgagaaggtggaacggccgacaggtctctgtcctgcagcggccgctgacacgaccccaggacagcggaaatgagacccaggctgcctgtcgggcacacggcggccgccccggcaccctggtgatcctgtgcagcacgaggtgcccagtagaggggtgggcagggcagggcagggcagggcaggacctggggacgcggccgcagcaggtgggctccagccaggaaggagccacgagtggggtggggtctgctcgctgggctggaggcagggagggcacctcggctccaacatcccagccggggacccggccagaggaaggggctgcgaggatgtcttccaagcatctctttgctcttggaaccacgtggcgaagctttctgaaagcagaccagactgcagcaccacggtttctattgtgaagggatttttccagaaggagtgggaccttgcgaacggggatgtggacacagggtggcttctgatcaagcccagggccctgtgcagccaagtctcatggtcacgccgtcccggccggaccagcctgccccccacgcggacccggggaaggcaggggtcccatgaccagcacgagaacgactgactcgccttgacctcaagccacctccaggaccaggggcagacggcccggcaccacgaggaggaggcagagccccgggcagggcccacttgccacttcacctcgccagagagccgggcagatggcggcacgcgactcttggggcctgacggccccgagcccagacgctctcggggctttggccagactcgcagggctgctgagccaagcagaggctacaaagctccacgggcttccaggcaaccgctagagacacgtgggcacagctcgcagagccggtgacccacgggccagatgggaacctccctcctgtgccccctcaaggcgtctcccggaacggtggcctcccacggcgctggacggggctcggggcaggcccgaggcagcacggcgcccacaccgtccactggccgcttggaaggattcagccaacaccaccccaggcccgctgcacagccggggtgggggctgagacgtccctcaggggtccccagtcccagcctgatgctgaagttacaggacggcccaggggcgcccaggggacagggggagctgcggccctccacgaatgaggacagagggtccctgacccccacgcacggccggcaggtctcggtccacgcagagcagtgagcggctgcccgccagcccgctctcggccgggcaccaccttcacaacccctgggaactctgtccccacgtgggcagcacgggcctgggtctgggcagcaggctgggcccagccacacacgcgggggtcacaagaggtgcccggtggggtcgggcaaccaagggctcacactaggggcctggcacccgggctgcagtaggccaggcggcataatccccgtgggacatagagaaccacgacgtcctgagttcccgcaccctccccaccgctcacccaccccacacacccacatgagccgccagggcactgtctggactcccccaggccctctgggacagaaagcaaccagaagaaaagggaacttcaggaagcagccgggccacccggtttcaatcccattcttagtgtgcagggctgtgggcaggaaggtcgtgcctccagggaccagacacccacccgaggaagcagggctgcaggcacagagcacagacacaaagagccacatgcacctggggccagagcaggaggggcccggagcccggggcagcaggggagccacctcctcggcccccacactcggtgcgtggggttcacgaagaccaggggcaggggcagcggaggggggacgaggaggggagaccccacagcagggctgcagccgacccagcccagggaacacagcctgcacgcgggtcatgtcgcccgcccagcacagggaccagggagccctggcaggggcacgcccacgccaacagaggtggtgccccagaggccagaggaggtcggtggggccgccaggcctctgcggaacatgatgggctggggtcagaggcctgagggatctgctcggccaggttgctggaggcacagggctgggcaggggctgaggacgaggccaggatggacaggtgggtctgggcggaggactcagggaagagacggacacagcgcaggggacaaagaggtcacctggggttgcagggtcctgtcccggctgccggagcccgcttgagccccagggcaggtatgaaagcagtaactgtcgggtcccggggaaggagccagggccacgggcagcaggtgagccgggagcagagcaggcagggcgggccgggctcctgtgtgcccagagctggcagcacagggactgagcctaaagctccacaggccgcaggagggctgacgggagccaggggcctgagggcaggagcgggagccaggggctgggagctgggcgccagtgcagctgagccagcaactagagggagccagggaccagacaggctccaagggtggtcgcggcgagaagagggtgagctcagaagggtcaggagcccctccgggaggggaccaggtgggacagaggctctgccagggggacgctacccagagagggtgggcacccagggctccgggaacaaaggccagccctgggaaagaaaggaagccaggagtggagctagacaaggctgagggttcgggggcaggaagaggggtggtgacactgaggcccaggagccccagggaaaggggcaggtaggctgcaggtgagcagggaaccgggagggcggaaaagctgtccttagagctgctgggaacacatagagctggtgcaggtgaggaggggttgggagggcaagggggctgaacctgaagctcctcggaactggtagagcttgtgcagatgagcaggggctgggagggcagggggcagccctgggaccacctgagagctgtagatcttgggcaggtgagcacaaactggtagggcaggggaagaacctgcagttcctgggagcaggaggagcttgtgcaggtgagcaggggctggagggcagggggctaaacctggagctattgggaagaggttaggcttgtgcaggcgagcagggggtgggagggcgagggtcagtcctgggagccctttcgagccaatagagcccaggcaggtgagcaggggcttggcgggcagggagcatccctgggatgagtttgtgcaggtgagcagggggtgggagtgtagggagcagtcctagcaactcctgggagctggtagagcttgggcaggtgaacaggggctggaagtgcagggggcacatctgggaggccctgggagcaattagcgctctggcaggtgagtaggggctcggagggcagggagcagccctgggagctccttggagcaggtagagcttgggcaggtgagcagggggtgggagggcagggggcagtcctagcaactcctgggagcttgtagagcttgggcaggtgagaacgagctggtagggcaggggaatgaacctggagttactgggtcaggaggagcatgtgcaggtgagcaggggctggagggcagggggctaaacctggagctattgggaagaggttaggcttgtgcaggcaagcagggggtgggagagcaagggtcagtcctgggagcccttgcgagccggtagagcccaggcaggtgagcaggggctgggcgggcagggagcagtcctagcaactcctgggagcaggtagagcttgtgcaggtgagcagggggtgggagggtagggagcagtcctgggaactcctgcgagcaggtagagcttgtgcaggtgggcaggggtgggagggcagggggctgaacctggagctcctcagaactggtagagcttgtgcagatgagcagtggctgggagggcagggggcagccctgggagcaatagaccttgggcaggtgagaacgagctggtagggcagtggaatgaacctggagttactgggtcaagaagagcttgtgcaggcaagcaggggctgaagggcagggggctaaacctggagctattgggaagaggttaggcttgtgcaggcgagcagggggtgggagggcaagggtcagtcctgggagcccttgcgagccggtagagcccaggcaggtgagcaggggctgggagggcagggagcatccctgggacctcttgggagcaggtagagcttgtgcaggtgagcagggggtgggagggtagggagccgtcctagcaactcctgaatctggtagagcttgcggaggtgaacaggggctgggagtgcagggggcagtcctgggagcccctgggagtaattagagcttgggcaggtgagcaggagcttggagtctagggggctaaactggagctattgggaagagtttaggcttgttcaggtgagcaaggggtgggagtagagggggcattcctaggagctcccgcgagttggtcaatctcgggcaggtaagcaggggctcagaggcctggagcagccctgggaccatctgggagcagggcgagcttgggcaggtgagccgggccaggggggatgcaggacagggcaggggcaacagagtgggggtgagctcaggggagccccaggctcgagggaggggccgatatagggctagccaggctggaaagtgggctcctggggggaaggtccccaggactgcgggggcaggggcagggctgaacagacgccatgggtcacagggatcgggccaacgggccataccctgttccagcaaagtgagtggacatgggacagagcagctggagtcctaagtgagaacagcggcacagggcagtgcacatggtcggggggtcgaggggctcatctgggcctgagctggggaagcaggcaatggctgaacagaacaggggggagaggaccggcccaaccgggcaggggcgcatggggtgctggcgccccgtgggagaggaaggccaggggcagggctcggctgcagcagtgctgccaggcaggctggcagcgggaagggcaggagcagagggagggctctggcctcagcaggcagctgggaggcccagagctgggctgcaggggccggggctctggggaaagctgcctcgggtgagctcagggccaagccaggagccccggtggaggagggaccggtgggcccagaggcccagatttggagcctgggccagggccggagggatggcaggggaggggcctcggaggagactacctgccctcgcccagggaaaggggcatgttggtgccagggaccaacgcgcacgggccttgactgcagagctgacccagaggaacggggcctggagagcaaacagcaggccaggaccaggggccaggagcagagcatgggcagtgggtggcagtgggtggcagtgtccctgccaggctggggaagcaggacggtggcctcaggggaccagcagaagctggccaggaacacaggccacccgctggtcgggccaggcacccacaggggctctgggccctggcgggctctcatccaggcggtcacccagctctgaccaggcagctgcgcccagaacctgcagctgcaatggcagcgagctgggcggctggtctgccgactttctggaagcaagtgggtgctgggcgcagcggccccgctgttctgagggcccgattcgctgcccgccccacaaggaacaaggccctggcggtcacgcagcctcctctcttccag

SEQ ID NO: 5 - DNA, 337 bp, Bos taurus
tccactagggtggccaggcacagtcaccagagggggagggctcgggtgcaggggtgcgggagggtgggggaggcagcggtgtttgtgtcctcttgtttttctctttcttctcaagccccctgcacctcatcacctgctgaaacatccaaaatagctctaggtggctactgagtcattgcgagcacagcccaacccaggtgtcccagccaggctgctcttctgagaatcgggccccaaaaccgagacctggccaggtgggcctggggcctgggcccggggccaaagcccaggggagtcctacgggggcagtgagttccccaaggcctggagagggccc

SEQ ID NO: 6 - DNA, 246 bp, Bos taurus
ccccaccccctaggcaggtgcgaggccctctcagtttcccccaggttactcatttggggcacactcagccttgcagggcatgcaaatggctgtttgttccacactgaaaaacatgtctaagcctctgtggttatttccagaaatagcctacgcccacgccccacctgcagccccagctctgaccctccagagtgccaggctggcctggagctcaggattcgggggaccctgcaccccctgccccag

SEQ ID NO: 7 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 8 - protein, 123 aa, Homo sapiens
MACRCLSFLLMGTFLSVSQTVLAQLDALLVFPGQVAQLSCTLSPQHVTIRDYGVSWYQQRAGSAPRYLLYYRSEEDHHRPADIPDRFSAAKDEAHNACVLTISPVQPEDDADYYCSVGYGFSP

SEQ ID NO: 9 - DNA, 177 bp, Homo sapiens
gtcacatctggactcccccaggccctctgagacagaaaacactcagaagaaaagggaacttcaggaagcaagtcgcgtcaccaggtttcattcctgttcttagtcttcacagcactgggggaagggccctcacgcctcctgggactggtgaccaagtcccagggagcagggctgcag

SEQ ID NO: 10 - protein, 476 aa, Bos taurus
EGESHPRVFPLVSCVSSPSDESTVALGCLARDFVPNSVSFSWKFNNSTVSSERFWTFPEVLRDGLWSASSQVVLPSSSAFQGPDDYLVCEVQHPKGGKTVGTVRVIATKAEVLSPVVSVFVPPRNSLSGDGNSKSSLICQATDFSPKQISLSWFRDGKRIVSGISEGQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 11 - protein, 471 aa, Equus ferus
ESTKTPDLFPLVSCGPSLDESLVAVGCLARDFLPNVITFSWNYQNNTVVRSQDIKNFPSVLREGKYTASSQVLLPSGDVPLVCTVNHSNGNKKVEVRPQVLIQDESPNVTVFIPPRDAFTGPGQRTSRLVCQATGFSPKEISVSWLRDGKPVESGFTTEEVQPQNKESWPVTYKVTSMLTITESDWLNQKVFTCHVEHQQGVFQKNVSSMCSPNSPVPIKIFAIPPSFAGIFLTKSAKLSCQVTNLGTYDSLSISWTRQNGEILKTHTNISESHPNGTFSALGEATICVEDWESGDDYICTVTHTDLPFPLKQAISRPDAVAKHPPSVYVLPPTREQLSLRESASVTCLVKGFSPPDVFVQWLQKGQPLSSDKYVTSAPMPEPQAPGLYFVHSILTVSEEDWSSGETYTCVVGHEALPHVVTERTVDKSTEGEVSAEEEGFENLSAMASTFIVLFLLSLFYSTTVTLFKVK

SEQ ID NO: 12 - protein, 476 aa, Ovis aries
ESESHPKVFPLVSCVSSPSDENTVALGCLARDFVPNSVSFSWKFNNSTVSSERFWTFPEVLRDGLWSASSQVALHSSSTFQGTDGYLVCEVQHPKGGKTVGTVMVVAPKVEVLSPVVSVFVPPCNSLSGNGNSKSSLICQATDFSPKQISLSWFRDGKRIVSDISEGQVETVQSSPTTYRAYSVLTITEREWLSQSAYTCQVEHNKETFQKNASSSCDATPPSPIGVFTIPPSFADIFLTKSAKLSCLVTNLASYDGLNISWSHQNGKALETHTYFERHLNDTFSARGEASVCSEDWESGEEYTCTVAHLDLPFPEKSAISKPKDVAMKPPSVYVLPPTREQLSLRESASVTCLVKGFAPADVFVQWLQKGEPVAKSKYVTSSPAPEPQDPSAYFVHSILTVTEEDWSKGETYTCVVGHEALPHMVTERTVDKSTEGEVSAEEEGFENLNTMASTFIVLFLLSLFYSTTVTLFKVK

SEQ ID NO: 13 - protein, 477 aa, Sus scrofa
ESQSAPNLFPLVSCVSPPSDESLVALGCLARDFLPSSVTFSWNYKNSSKVSSQNIQDFPSVLRGGKYLASSRVLLPSVSIPQDPEAFLVCEVQHPSGTKSVSISGPVVEEQPPVLNIFVPTRESFSSTPQRTSKLICQASDFSPKQISMAWFRDGKRVVSGVSTGPVETLQSSPVTYRLHSMLTVTESEWLSQSVFTCQVEHKGLNYEKNASSLCTSNPNSPITVFAIAPSFAGIFLTKSAKLSCLVTGLVTRESLNISWTRQDGEVLKTSIVFSEIYANGTFGARGEASVCVEDWESGDRFTCTVTHTDLPSPLKQSVSKPRGIARHMPSVYVLPPAPEELSLQEWASVTCLVKGFSPADVFVQWLQKGEPVSADKYVTSAPVPEPEPKAPASYFVQSVLTVSAKDWSDGETYTCVVGHEALPHTVTERTVDKSTEGEVSAEEEGFENLNTMASTFIVLFLLSLFYSTTVTLFKVK

SEQ ID NO: 14 - protein, 471 aa, Artificial Sequence
  chimeric IgM
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

(19) United States
(12) Patent Application Publication
(10) Pub. No.: US 2021/0246197 A1
(43) Pub. Date: Aug. 12, 2021

(54) ANTI-THYMOCYTE GLOBULIN

(71) Applicant: SAB, LLC., Sioux Falls, SD (US)

(72) Inventors: Eddie J. SULLIVAN, Sioux Falls, SD (US); Hua WU, Sioux Falls, SD (US); Christoph L. BAUSCH, Sioux Falls, SD (US)

(21) Appl. No.: 17/171,480

(22) Filed: Feb. 9, 2021

Related U.S. Application Data
(60) Provisional application No. 62/975,649, filed on Feb. 12, 2020.

(51) Int. Cl. C07K 16/18

(52) U.S. Cl. CPC C07K 16/18; C07K 2317/734; C07K 2317/10; C07K 2317/21

(57) ABSTRACT

Provided are human anti-thymocyte globulin (ATG) products, and methods of making and using the same. In particular, the disclosure provides an ungulate-derived polyclonal immunoglobulin, comprising a population of fully human or substantially human immunoglobulins. The population of fully human or substantially human immunoglobulins specifically binds human thymocytes, T cells, B cells, and/or monocytes. Such compositions may be made by immunization of transgenic animals having a human Ig locus with human thymocyte. This method generates polyclonal immunoglobulin with yield, purity, and antigen specificity that enable use of this product in medical applications.

### CROSS-REFERENCE TO RELATED APPLICATIONS

This application claims priority to U.S. Application No. 62/975,649, filed Feb. 12, 2020, which is incorporated by reference herein in its entirety.

### INCORPORATION BY REFERENCE OF SEQUENCE LISTING

The present application is being filed with a Sequence Listing in electronic format. The Sequence Listing is provided as a file entitled SABB_001_01US_SeqList_ST25.txt, created on Feb. 8, 2021 and is 70 kilobytes in size. The information in electronic format of the Sequence Listing is incorporated by reference in its entirety.

### TECHNICAL FIELD

The invention relates generally to methods of making anti-thymocyte globulin for biomedical applications.

### BACKGROUND

ATG (anti-thymocyte globulin) is a polyclonal immunoglobulin that is FDA-approved for use in organ transplantation. ATG is also used or in clinical trials for treatment of graft-versus-host disease and type 1 diabetes (T1D). In T1D, ATG is used as monotherapy to preserve β cell function and as an immunosuppressive for nonmyeloablative hematopoietic stem cell transplantation.

Current ATG products are produced by immunization of rabbits or horses to generate polyclonal xenobiotic immunoglobulin. ATG therapy puts patients at risk for serum sickness as the recipient's immune system reacts to the xenobiotic immunoglobulin in the ATG. The immune response to ATG also renders redosing problematic. Furthermore, serum sickness in T1D cannot be managed with glucocorticoids because glucocorticoids impair the function of the β cells of the patient.

Thus, there remains a need in the art for improved methods of producing ATG, with associated compositions and methods of use.

### SUMMARY

The present disclosure relates generally to anti-thymocyte globulin (ATG) produced in a transgenic animal having a fully human (or at least partially human) immunoglobulin locus. The resulting composition comprises fully human (or substantially human) immunoglobulin. Surprisingly, immunization of the transgenic animal with human thymocytes generates ATG having potency as greater or greater than reference ATG products. Thus, the methods disclosed herein produce ATG in sufficient yield and potency to permit manufacturing of a human ATG product that solves the problem of serum sickness due to the xenobiotic immunoglobulin.

In one aspect, the disclosure provides an ungulate polyclonal immunoglobulin composition, comprising a population of fully human or substantially human immunoglobulins. The population of fully human or substantially human immunoglobulins specifically binds human thymocytes, T cells, B cells, and/or monocytes.

In another aspect, the disclosure provides a composition produced by immunizing a transgenic ungulate with human thymocytes. The composition comprises a population of fully human or substantially human immunoglobulins. The population of fully human or substantially human immunoglobulins specifically binds human thymocytes, T cells, B cells, and/or monocytes.

In yet another aspect, the disclosure provides a method of producing anti-thymocyte globulin (ATG), comprising administering human thymocytes to a transgenic ungulate. The genome of the transgenic ungulate comprises a human immunoglobulin locus. The transgenic ungulate produces a polyclonal immunoglobulin comprising anti-thymocyte globulin (ATG).

In a further aspect, the disclosure provides a method of providing anti-thymocyte globulin (ATG) treatment to a subject in need thereof, comprising administering to the subject: i) a polyclonal immunoglobulin composition according to the disclosure; ii) a composition according to the disclosure; or iii) a polyclonal immunoglobulin composition produced according to the disclosure. The method provides an effective amount of anti-thymocyte globulin (ATG) to the subject.

In yet a further aspect, the disclosure provides a pharmaceutical composition, comprising a population of fully human or substantially human immunoglobulins, and one or more pharmaceutically acceptable excipients. The population of fully human or substantially human immunoglobulins specifically binds human thymocytes, T cells, B cells, and/or monocytes.

Additional embodiments, features, and advantages of the invention will be apparent from the following detailed description and through practice of the invention.

### BRIEF DESCRIPTION OF THE DRAWINGS

FIGS. 1A-1H show construction of the isHAC and isKcHACΔ vectors.

FIG. 1A shows a flow of the isHAC and isKcHACΔ vector construction. The bovinizing vector pCC1BAC-isHAC is a BAC-based one (backbone is pCC1BAC vector), consisting of 10.5 kb and 2 kb of genomic DNA as a long and short arm, respectively, 9.7 kb of the bovine genomic DNA covering the bovine I_{γ1}-S_{γ1} and its surrounding region to replace the human corresponding 6.8 kb of I_{γ1}-S_{γ1} region, the chicken β-actin promoter-driven neo gene flanked by FRT sequence and DT-A gene. After the targeted bovinization, the neo cassette is removed by FLP introduction.

FIG. 1B shows detailed information on the targeting vector pCC1BAC-isHAC. The 2 kb of Afe I-Bam HI fragment and 10.5 kb of Apa I-Hpa I fragment for a short arm and long arm were obtained from clone h10 and clone h18/h20, respectively, derived from λ, phage genomic library constructed from CHO cells containing the κHAC by screening using a probe around the human I_{γ1}-S_{γ1} region. The 9.7 kb fragment (5′ end through Bsu36 I) was obtained from clone b42 derived from the λ phage bovine genomic library.

FIG. 1C shows senotyping of the bovinized I_{γ1}-S_{γ1} region. Five sets of genomic PCR were implemented, as indicated. iscont1-F1/R1 is a positive PCR specific to the homologous recombination. iscont1-F1×hIgG1-R10 is a negative PCR that is prohibited by the presence of the neo cassette. isHAC-Sw-dig-F5/R3 and isHAC-TM-dig-F3/R2 are for structural integrity check of their corresponding region, digested by Bam HI+Pvu II and Age I, Sma I or Pvu II, respectively. bNeo 5′-R×bIgG1-5′-seq-R6 is to confirm the presence of FRT sequence.

FIG. 1D shows genotyping after the FLP-FRT deletion of the neo cassette.

FIG. 1E shows extensive genomic PCR for genotyping of the isHAC vector. Location of each genomic PCR primer pair is depicted in relation to the isHAC vector structure.

FIG. 1F shows CGH analysis among three different CHO clones containing the isHAC vector. DNA from isC1-133 was used as a reference. There was no apparent structural difference of the isHAC among the three cell lines.

FIG. 1G shows extensive genomic PCR for genotyping of the isKcHACΔ vector. Location of each genomic PCR primer pair is depicted in relation to the isKcHACΔ vector structure.

FIG. 1H shows CGH analysis among three different CHO clones containing the isKcHACΔ vector. DNA from isKCDC15-8 was used as a reference. There was no apparent structural difference of the isKcHACΔ among the three cell lines.

FIG. 2 shows flow cytometry assessment of binding of a TcB-derived ATG product to human PBMCs.

FIGS. 3A-3B show levels of regulatory T (Treg) cells treated with horse (Ho-ATG), rabbit (Rb-ATG), or TcB (SAB-ATG) products. FIG. 3A shows percentage of CD4+CD25+ Foxp3+ cells.

FIG. 3B shows the percentage of CD4+CD25+ Foxp3+ cells relative to the amount of immunoglobulin G. (** indicates two-tailed p-value less than 0.001).

FIGS. 4A-4B show levels of activated conventional T (Tconv) cells treated with horse (Ho-ATG), rabbit (Rb-ATG), or TcB (SAB-ATG) products. FIG. 4A shows percentage of CD4+CD25+ Foxp3− cells. FIG. 4B shows the percentage of CD4+CD25+ Foxp3− cells relative to the amount of immunoglobulin G. (two-tailed p-values: **=less than 0.05; **=less than 0.001; ***=less than 0.0001).

FIGS. 5A-5B show levels of naïve conventional T (Tconv) cells treated with horse (Ho-ATG), rabbit (Rb-ATG), or TcB (SAB-ATG) products. FIG. 5A shows percentage of CD4+CD25− Foxp3-cells. FIG. 5B shows the percentage of CD4+CD25− Foxp3− cells relative to the amount of immunoglobulin G. (two-tailed p-values: **=less than 0.05; **=less than 0.001; ***=less than 0.0001).

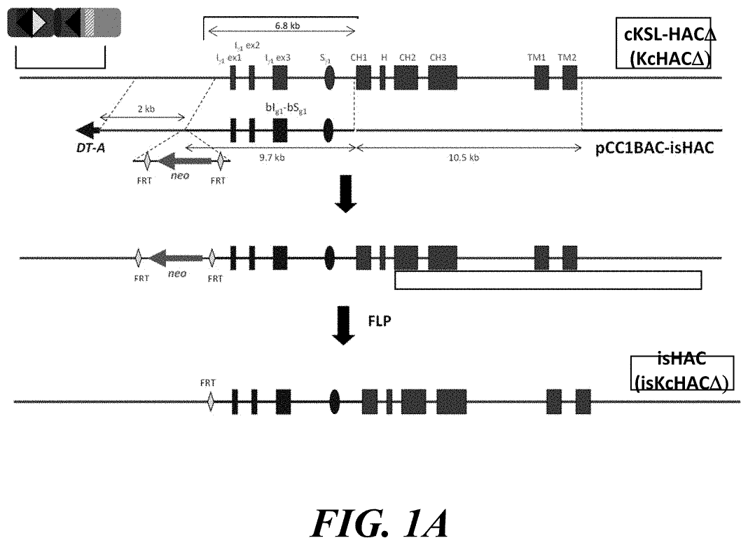

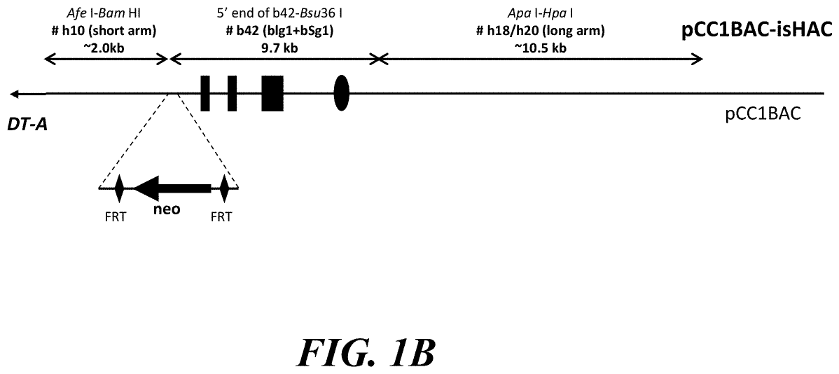

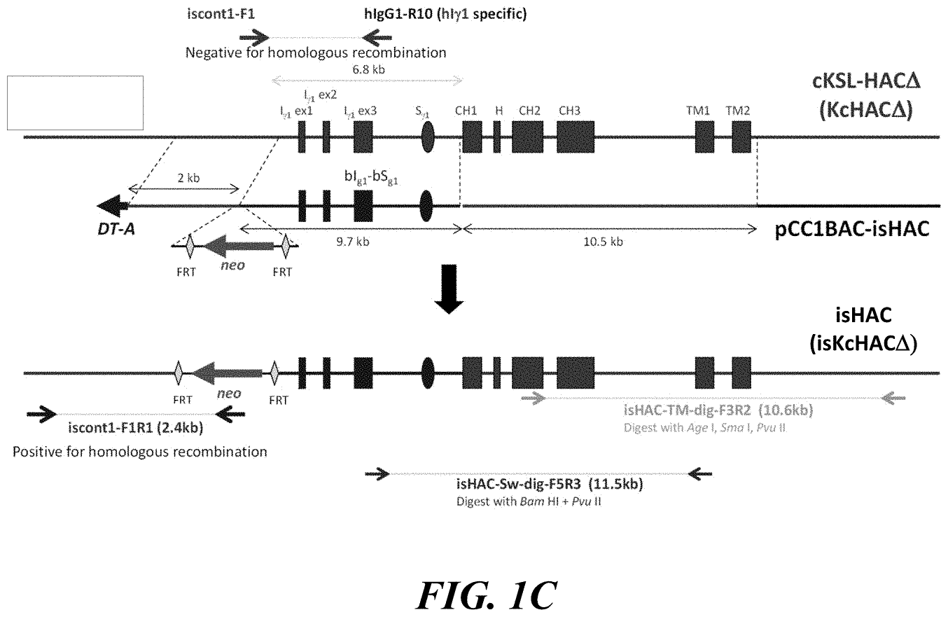

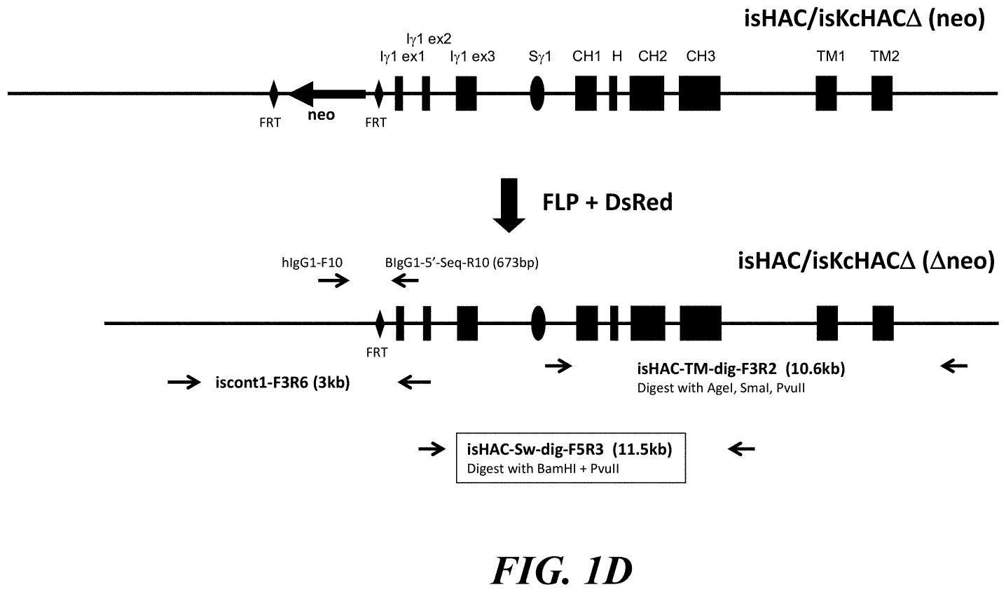

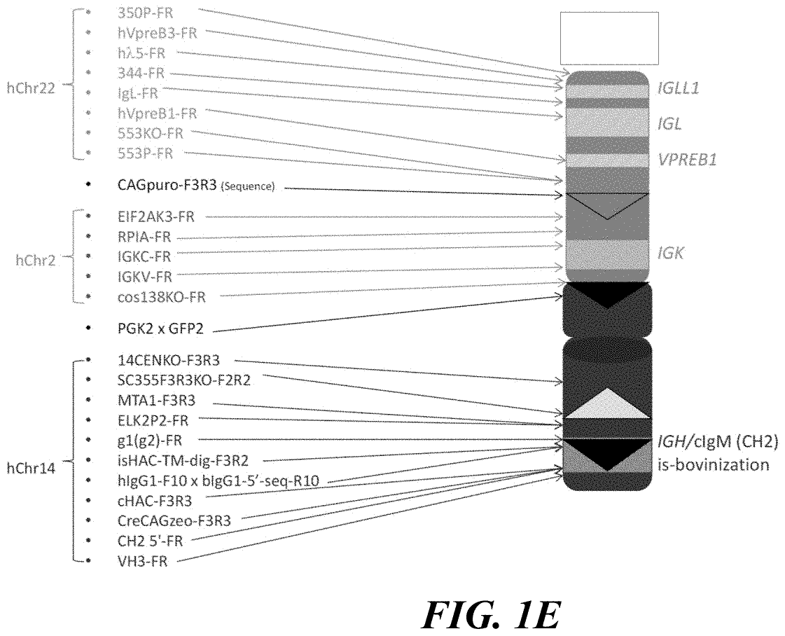

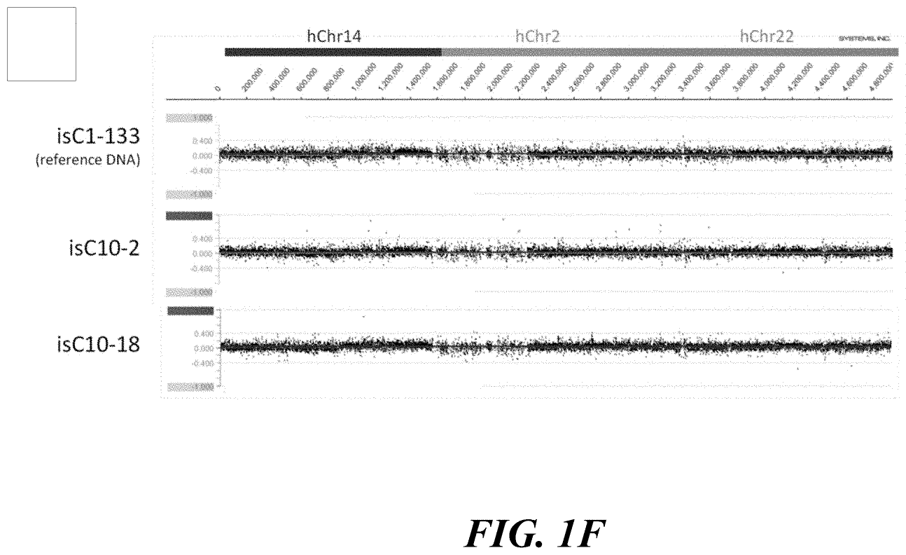

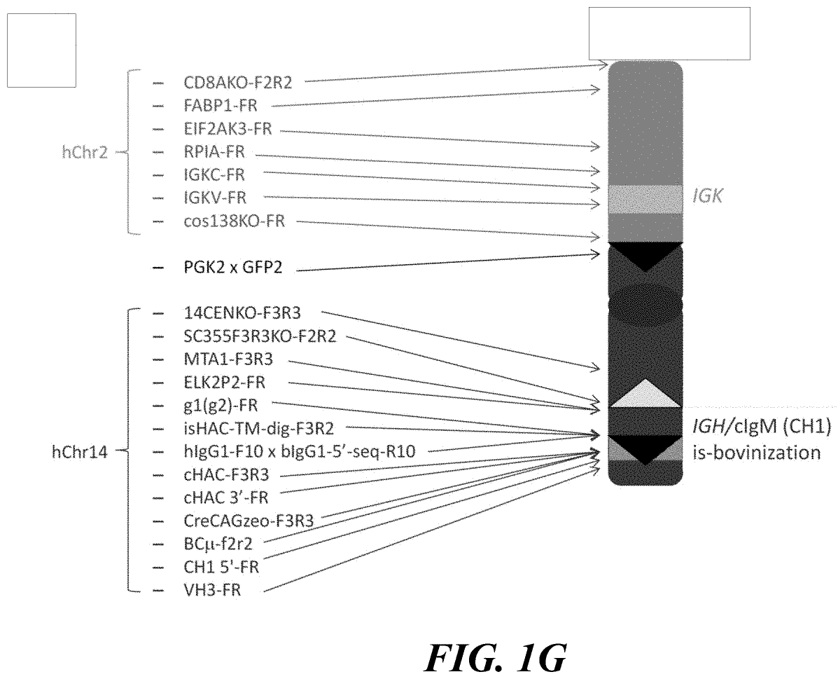

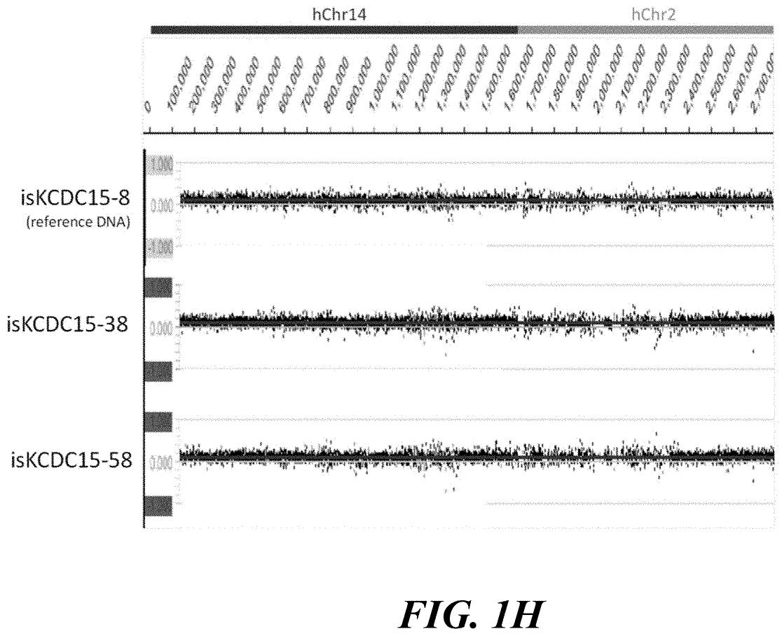

### DETAILED DESCRIPTION

The present inventors have developed a human ATG product that overcomes limitations of animal ATGs. Transgenic animals with the endogenous Ig locus replaced by a human artificial chromosome encoding a human Ig locus express fully human polyclonal antibodies. Immunization of such a transgenic animal with human thymocytes generates polyclonal immunoglobulin with yield, purity, and antigen specificity that enable use of this product in medical applications. Various embodiments of the invention are provided in the description that follows.

### Definitions

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

The term “ungulate” refers to any suitable ungulate, including but not limited to bovine, pig, horse, donkey, zebra, deer, oxen, goats, sheep, and antelope.

The term “transgenic” means the cells of the ungulate comprise one or more polynucleotides encoding exogenous gene(s)(e.g. an immunoglobulin locus). Such as polynucleotide may be a portion of an artificial chromosome. Alternatively, or in addition to an artificial chromosome, one or more polypolynucleotides encoding exogenous gene(s) may be integrated into the genome of the cells of the ungulate.

The terms “polyclonal” or “polyclonal serum” or “polyclonal plasma” or “polyclonal immunoglobulin” refer to a population of immunoglobulins having shared constant regions but diverse variable regions. The term polyclonal does not, however, exclude immunoglobulins derived from a single B cell precursor or single recombination event, as may be the case when a dominant immune response is generated. A polyclonal serum or plasma contains soluble forms (e.g., IgG) of the population of immunoglobulins. The term “purified polyclonal immunoglobulin” refers to polyclonal immunoglobulin purified by serum or plasma. Methods of purifying polyclonal immunoglobulin include, without limitation, caprylic acid fractionation and adsorption with red blood cells (RBCs).

A “population” of immunoglobulins refers to immunoglobulins having diverse sequences, as opposed to a sample having multiple copies of a single immunoglobulin. Similarly stated, the term population excludes immunoglobulins secreted from a single B cell, plasma cell, or hybridoma in culture, or from a host cells transduced or transformed with recombinant polynucleotide(s) encoding a single pair of heavy and light chain immunoglobulin sequences.

The term “immunoglobulin” refers to a protein complex at least two heavy and at least two light chains in 1:1 ratio, including any of the five classes of immunoglobulin—IgM, IgG, IgA, IgD, IgE. In variations, the immunoglobulin is engineered in any of various ways known in the art or prospectively discovered, including, without limitation, mutations to change glycosylation patterns and/or to increase or decrease complement dependent cytoxocity.

An immunoglobulin is “fully human or substantially human” when the protein sequence of the immunoglobulin is sufficiently similar to the sequence of a native human immunoglobulin that, when administered to a subject, the immunoglobulin generates an anti-immunoglobulin immune response similar to, or not significantly worse, that the immune reaction to native human immunoglobulin. A fully human immunoglobulin will comprise one or more substitutions, insertions, to deletions in variable regions, consistent with recombination, selection, and affinity maturation of the immunoglobulin sequence. In variations, the fully human or substantially human immunoglobulin is engineered in any of various ways known in the art or prospectively discovered, including, without limitation, mutations to change glycosylation patterns and/or to increase or decrease complement dependent cytoxocity.

The terms “thymocytes”, “T cells”, “B cells”, and “monocytes” are given there ordinary meaning in the art. Thymocytes are hematopoietic progenitor cells present in the thymus. In the methods of the disclosure, administering (human) thymocytes may refer, in some embodiments, to administering a mixed population of cells that include thymocytes, provided thymocytes are present in sufficient quantity and purity to generate an anti-thymocyte immune response in the transgenic ungulate. In variations of the methods of the disclosure, non-human thymocytes are used, such as for example thymocytes of a non-human primate.

The percentage of an immunoglobulin (e.g., immunoglobulin that specifically binds human thymocytes) “by mass of total immunoglobulin” refers to the concentration of a target immunoglobulin population divided by the concentration of total immunoglobulin in a sample, multiplied by 100. The concentration of target immunoglobulin can be determined by, for example, affinity purification of target immunoglobulin (e.g. on affinity column comprising thymocytes or thymocyte cell membranes) followed by concentration determination.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

The terms “immunization” and “immunizing” refer to administering a composition to a subject (e.g., a transgenic ungulate) in an amount sufficient to elicit, after one or more administering steps, a desired immune response (e.g., a polyclonal immunoglobulin response specific to thymocytes). Administration may be by intramuscular injection, intravenous injection, intraperitoneal injection, or any other suitable route. Immunization may comprise between one and ten, or more administrations (e.g. injections) of the composition, such as 1, 2, 3, 4, 5, 6, 7, 8, 9, 10, or more administrations. The first administration may elicit no detectable immune response as generally each subsequence administration will boost the immune response generated by prior administrations.

The term “target antigen” refers to any antigen use to elicit a desired immune response. The target antigen used to generate an ATG product may be thymocyte cells, cells sharing one or more endogenous protein markers with thymocytes, cells recombinantly expressing one or more thymocyte proteins, recombinant thymocyte proteins, or nucleic acids that encoding thymocyte proteins (e.g. RNA, linear DNA, or plasmid DNA).

The term “purify” refers to separating a target cell or molecule (e.g. a population of immunoglobulins, thymocytes) from other substances present in a composition. Immunoglobulins may be purified by fractionation of plasma, by affinity (e.g. protein A or protein G binding, or other capture molecule), by charge (e.g. ion-exchange chromatography, by size (e.g. size exclusion chromatograph), or otherwise. Purifying a population of immunoglobulins may comprise treating a composition comprising the population of immunoglobulins with one or more of acids, bases, salts, enzymes, heat, cold, coagulation factors, or other suitable agents. Purifying may further include adsorption of a composition comprising a target cell or molecule and an impurity onto non-target cells or molecules (e.g., red blood cells) to partially or completely remove the impurity. Purifying may further include pre-treatment of serum or plasma, e.g., caprylic acid fractionation.

The terms “treating” and “treatment” refer to one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “pharmaceutically acceptable” means biologically or pharmacologically compatible for in vivo use in animals or humans, and can mean approved by a regulatory agency of the Federal or a state government or listed in the U.S. Pharmacopeia or other generally recognized pharmacopeia for use in animals, and more particularly in humans.

The term “hyperimmunized” refers to immunization regimen that generates an immune response to the subject greater than required to produce an desired titer (e.g. a binding titer) after dilution of the immunoglobulin produced by the subject. For example, if a desired titer is 1:100, one may hyperimmunize an animal by a prime immunization followed by one, two, three or more boost immunizations to produce a 1:1,000 titer, or greater titer, in the subject-so that immunoglobulin produced by the subject may be diluted in the production of a biotherapeutic in order to give a desired titer in the biotherapeutic.

An immunoglobulin is “specific to” or “specifically binds” (used interchangeably herein) to a target (e.g., thymocytes or a thymocyte antigen) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An immunoglobulin “specifically binds” to a particular cell or substance if it binds with greater affinity, avidity, more readily, and/or with greater duration than it binds to alternative particular cell or substance. For example, an immunoglobulin that specifically or preferentially binds to thymocyte is an immunoglobulin that binds thymocytes with greater affinity, avidity, more readily, and/or with greater duration than it binds to other cells. An immunoglobulin that specifically to a first cell or substance may or may not specifically or preferentially bind to a second cell or substance. As such, “specific binding” does not necessarily require (although it can include) exclusive binding. Generally, but not necessarily, reference to binding means specific binding.

The term “HAC vector” means a vector which comprises at least a human chromosome-derived centromere sequence, a telomere sequence, and a replication origin, and may contain any other sequences as desired for a given application. When present in a host cell, the HAC vector exists independently from a host cell chromosome the nucleus. Any suitable methods can be used to prepare HAC vectors and to insert nucleic acids of interest into the HAC, including but not limited to those described in the examples that follow. The HAC vector is a double stranded DNA vector, as is known to those of skill in the art.

### EMBODIMENTS

Provided are methods of producing anti-thymocyte globulin (ATG), comprising administering human thymocytes to a transgenic ungulate. Thymocytes are hematopoietic progenitor cells present in the thymus. They are available from various sources, including pediatric and young adult cardiac surgeries where thymus tissue must be removed from the patient and would normally be discarded. The thymocytes may be live human thymocytes, as live human thymocytes better preserve the conformation of surface antigens. In some embodiments, the method comprises administering an effective amount of human thymocytes. In embodiments, the effective amount is at least about 1×10⁸, at least about 5×10⁸, at least about 1×10⁹, at least about 5×10⁹, at least about 1×10¹⁰, or at least about 5×10¹¹ thymocytes.

In a variation, non-human thymocytes are used (e.g., thymocytes of a domesticated animal such as a dog, cat, sheep, etc.). The transgenic ungulate may in such cases comprise an artificial chromosome encoding an Ig locus of the non-human species such that antibodies of that species are generated.

In some embodiments, the thymocytes are administered before, during, or after administration of one or more adjuvants. In some embodiments, the thymocytes and one or more adjuvants are administered together in a single composition, comprising optionally one or more pharmaceutically acceptable excipients.

Illustrative adjuvants include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. Further illustrative adjuvants include, but are not limited to, Adju-Phos, Adjumerlm, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, MONTANIDE™ ISA-25, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, ISA-25/Quil-A, Rehydragel HPA, Rehydragel LV, RIBI, Ribilike adjuvant system (MPL, TMD, CWS), S-28463, SAB-adj-1, SAB-adj-2, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes.

The immunization may be carried out by administering human thymocytes with, for example, a complete Freund's adjuvant or an appropriate adjuvant such as an aluminum hydroxide gel, and pertussis bacteria vaccine, subcutaneously, intravenously, or intraperitoneally into a transgenic ungulate. In one embodiment, the immunization comprises hyperimmunization. In various embodiments, the human thymocytes are administered once to 10 times every 1 to 4 weeks after the first administration. After 1 to 14 days from each administration, blood is collected from the animal to measure the antibody value of the serum.

In some embodiments, the human thymocytes are administered 3, 4, 5, 6 or more times. Administration of the human thymocytes may be performed, e.g., every 1-2 weeks, 2-3 weeks, 3-4 weeks, 4-5 weeks, 5-6 weeks, or 6-7 weeks, or longer intervals, e.g., every 1 week, 2 weeks, 3 weeks, 4 weeks, 5 weeks or 6 weeks. After each immunization, serum and/or plasma may be harvested from the transgenic ungulate one or more times. For example, the method may be including performing controls bleeds two or three times at intervals about 7-14 days.

In some embodiments, antigen used to generate an ATG product may be—rather than thymocytes-cells sharing one or more endogenous protein markers with thymocytes, cells recombinantly expressing one or more thymocyte proteins, recombinant thymocyte proteins, or nucleic acids that encoding thymocyte proteins (e.g. RNA, linear DNA, or plasmid DNA).

In embodiments of the methods of the disclosure, the genome of the transgenic ungulate comprises a human immunoglobulin locus. Illustrative methods are provided in U.S. Pat. Nos. 9,902,970; 9,315,824; 7,652,192; and 7,429,690; and 7,253,334, the disclosure of which are incorporated by reference herein for all purposes. Further illustrative methods are provided by Kuroiwa, Y., et al. (2009) Nat Biotechnol. 27(2):173-81, and Matsushita et al. (2015) PLoS ONE 10(6):e0130699.

The disclosure provides a human artificial chromosome (HAC) vector comprising genes encoding:

(a) one or more human antibody heavy chains, wherein each gene encoding an antibody heavy chain is operatively linked to a class switch regulatory element;

(b) one or more human antibody light chains; and

(c) one or more human antibody surrogate light chains, and/or an ungulate-derived IgM heavy chain constant region;

wherein at least one class switch regulatory element of the genes encoding the one or more human antibody heavy chains is replaced with an ungulate-derived class switch regulatory element.

The HAC vectors of the disclosure can be used, for example, for large-scale production of fully human antibodies by transgenic animals, as described for the methods of the invention. The HAC vector of the present disclosure comprises one or more genes encoding a human antibody heavy chain. Any human antibody heavy chain or combinations of human antibody heavy chains in combination may be encoded by one or more nucleic acids on the HAC. In various embodiments, 1, 2, 3, 4, 5, 6, 7, 8, or all 9 of human antibody heavy chains IgM, IgG1, IgG2, IgG3, IgG4, IgA1, IgA2, IgE and IgD may be encoded on the HAC in one or more copies. In one embodiment, the HAC comprises a human IgM antibody heavy chain encoding gene, alone or in combinations with 1, 2, 3, 4, 5, 6, 7, or the other 8 human antibody chain encoding genes. In one preferred embodiment, the HAC comprises a gene encoding at least a human IgG1 antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another embodiment, the HAC comprises a gene encoding at least a human IgA antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another preferred embodiment, the HAC comprises genes encoding all 9 antibody heavy chains, and more preferably where the gene encoding a human IgM antibody heavy chain has been chimerized to encode an ungulate-derived IgM heavy chain constant region. In another embodiment, the HAC may comprise a portion of human chromosome 14 that encodes the human antibody heavy chains. The variable region genes and the constant region genes of the human antibody heavy chain form a cluster and the human heavy chain locus is positioned at 14q32 on human chromosome 14. In one embodiment, the region of human chromosome 14 inserted in the HAC comprises the variable region and the constant region of the human antibody heavy chains from the 14q32 region of human chromosome 14.

In some embodiments of the HAC vectors of the present disclosure, at least one class switch regulatory element of the human antibody heavy chain encoding nucleic acid is replaced with an ungulate-derived class switch regulatory element. The class switch regulatory element refers to nucleic acid which is 5′ to an antibody heavy chain constant region. Each heavy chain constant region gene is operatively linked with (i.e. under control of) its own switch region, which is also associated with its own I-exons. Class switch regulatory elements regulate class switch recombination and determine Ig heavy chain isotype. Germline transcription of each heavy chain isotype is driven by the promoter/enhancer elements located just 5′ of the I-exons and those elements are cytokine or other activator-responsive. In a simple model of class switch, the specific activators and/or cytokines induce each heavy chain isotype germline transcription from its class switch regulatory element (i.e., activator/cytokine-responsive promoter and/or enhancer). Class switch is preceded by transcription of I-exons from each Ig heavy (IGH) locus-associated switch region. As each heavy chain constant region gene is linked with its own switch region.

Any suitable ungulate-derived class switch regulatory element can be used. For example, the human heavy chain gene isotypes listed below has the following class switch regulatory elements:

IgM: Iμ-Sμ,

IgG1: Iγ1-Sγ1,

IgG2: Iγ2-Sγ2,

IgG3: Iγ3-Sγ3,

IgG4: Iγ4-Sγ4,

IgA1: Iα1-Sα1,

IgA2: Iα2-Sα2, and

IgE: Iε-Sε.

In various embodiments, 1, more than 1, or all of the human antibody heavy chain genes on the HAC have their class switch regulatory element replaced with an ungulate-derived class switch regulatory element, including but not limited to ungulate Iμ-Sμ, Iγ-Sγ, Iα-Sα, or Iε-Sε, class switch regulatory elements. In one embodiment, an Iγ1-Sγ1 human class switch regulatory element for human IgG1 heavy chain encoding nucleic acid on the HAC (such as that in SEQ ID NO: 1) is replaced with an ungulate Iγ1-Sγ1 class switch regulatory element. Exemplary ungulate Iγ1-Sγ1 class regulatory switch elements include a bovine IgG1 Iγ1-Sγ1 class switch regulatory element (SEQ ID NO: 2), a horse Iγ1-Sγ1 class switch regulatory element (SEQ ID NO: 3), and a pig Iγ1-Sγ1 class switch regulatory element (SEQ ID: 4). However, it is not necessary to replace the human class switch regulatory element with an ungulate class switch regulatory element from the corresponding heavy chain isotype. Thus, for example, an Iγ3-Sγ3 human class switch regulatory element for human IgG3 heavy chain encoding nucleic acid on the HAC can be replaced with an ungulate Iγ1-Sγ1 class switch regulatory element. As will be apparent to those of skill in the art based on the teachings herein, any such combination can be used in the HACs of the disclosure.

In another embodiment, the HAC comprises at least one ungulate enhancer element to replace an enhancer element associated with one or more human antibody heavy chain constant region encoding nucleic acids on the HAC. There are two 3′ enhancer regions (Alpha 1 and Alpha 2) associated with human antibody heavy chain genes. Enhancer elements are 3′ to the heavy chain constant region and also help regulate class switch. Any suitable ungulate enhancer can be used, including but not limited to 3′Eα enhancers. Non-limiting examples of 3′ Eα enhancers that can be used include 3′Eα, 3′Eα1, and 3′Eα2. Exemplary 3′Eα enhancer elements from bovine that can be used in the HACs and replace the human enhancer include, but are not limited to bovine HS3 enhancer (SEQ ID NO: 5), bovine HS12 enhancer (SEQ ID NO: 6), and bovine enhancer HS4. This embodiment is particularly preferred in embodiments wherein the HAC comprises the variable region and the constant region of the human antibody heavy chains from the 14q32 region of human chromosome 14.

The HAC vectors of the present disclosure may comprise one or more genes encoding a human antibody light chain. Any suitable human antibody light chain-encoding genes can be used in the HAC vectors of the invention. The human antibody light chain includes two types of genes, i.e., the kappa/K chain gene and the lambda/L chain gene. In one embodiment, the HAC comprises genes encoding both kappa and lambda, in one or more copies. The variable region and constant region of the kappa chain are positioned at 2p11.2-2p12 of the human chromosome 2, and the lambda chain forms a cluster positioned at 22q11.2 of the human chromosome 22. Therefore, in one embodiment, the HAC vectors of the invention comprise a human chromosome 2 fragment containing the kappa chain gene cluster of the 2p11.2-2p12 region. In another embodiment, the HAC vectors of the present invention comprise a human chromosome 22 fragment containing the lambda chain gene cluster of the 22q11.2 region.

In another embodiment, the HAC vector comprises at least one gene encoding a human antibody surrogate light chain. The gene encoding a human antibody surrogate light chain refers to a gene encoding an transient antibody light chain which is associated with an antibody heavy chain produced by a gene reconstitution in the human pro-B cell to constitute the pre-B cell receptor (preBCR). Any suitable human antibody surrogate light chain encoding gene can be used, including but not limited to the VpreB1 (SEQ ID NO: 7), VpreB3 (SEQ ID NO: 8) and λ5 (also known as IgLL1, SEQ ID NO: 9) human antibody surrogate light chains, and combinations thereof. The VpreB gene and the λ5 gene are positioned within the human antibody lambda chain gene locus at 22q11.2 of the human chromosome 22. Therefore, in one embodiment the HAC may comprise the 22q11.2 region of human chromosome 22 containing the VpreB gene and the λ5 gene. The human VpreB gene of the present invention provides either or both of the VpreB1 gene (SEQ ID NO: 7) and the VpreB3 (SEQ ID NO: 8) gene and in one embodiment provides both of the VpreB1 gene and the VpreB3 gene.

In yet another embodiment, the HAC vector comprises a gene encoding an ungulate-derived IgM heavy chain constant region. In this embodiment, the IgM heavy chain constant region is expressed as a chimera with the human IgM antibody heavy chain variable region. Any suitable ungulate IgM heavy chain antibody constant region encoding nucleic acid can be used, including but not limited to bovine IgM, (SEQ ID NO: 10), horse IgM, (SEQ ID NO: 11), sheep IgM, (SEQ ID NO: 12), and pig IgM, (SEQ ID NO: 13). In one embodiment, the chimeric IgM comprises the sequence in SEQ ID NO: 14. Pre-BCR/BCR signaling through the IgM heavy chain molecule promotes proliferation and development of the B cell by interacting with the B cell membrane molecule Ig-alpha/Ig-beta to cause a signal transduction in cells. Transmembrane region and/or other constant region of IgM are considered to have important roles in the interaction with Ig-alpha/Ig-beta for signal transduction. Examples of the IgM heavy chain constant regions include nucleic acids encoding constant region domains such as CH1, CH2, CH3, and CH4, and the B-cell transmembrane and cytoplasmic domains such as TM1 and TM2. The nucleic acid encoding an ungulate-derived IgM heavy chain constant region which is comprised in the human artificial chromosome vector of the invention is not particularly limited so long as the region is in a range which may sufficiently induce the signal of the B-cell receptor or B-cell proliferation/development in the above-described IgM heavy chain constant region. In one embodiment, the nucleic acid encoding an ungulate-derived IgM heavy chain constant region provides a transmembrane and cytoplasmic TM1 domain and TM2 domain derived from an ungulate, and in other embodiments encodes the ungulate-derived CH2 domain, CH3 domain, CH4 domain, TM1 domain, and TM2 domain or the ungulate-derived CH1 domain, CH2 domain, CH3 domain, CH4 domain, TM1 domain, and TM2 domain.

In one embodiment, the gene encoding the IgM heavy chain constant region of the bovine is a gene encoding a bovine IgM heavy chain constant region which is included in an IGHM region at which a bovine endogenous IgM heavy chain gene is positioned (derived from IGHM) or a gene encoding a bovine IgM heavy chain constant region in an IGHML1 region (derived from IGHML1). In another embodiment, the gene encoding a bovine IgM heavy chain constant region is included in the IGHM region.

In a further embodiment, the HAC comprises a gene encoding a human antibody heavy chain comprises a gene encoding a human heavy chain (for example, a human IgG heavy chain, such as an IgG1 heavy chain), and wherein a transmembrane domain and an intracellular domain of a constant region of the human heavy chain gene are replaced with a transmembrane domain and an intracellular domain of an ungulate-derived heavy chain (for example, an ungulate IgG heavy chain, such as an IgG1 heavy chain), constant region gene. In one embodiment, gene encoding the transmembrane domain and the intracellular domain of an ungulate-derived (such as bovine) IgG (such as IgG1) heavy chain constant region are used to replace the corresponding regions of the human IgG heavy chain gene. In another embodiment, the gene encoding the TM1 and TM2 domains of an ungulate-derived (such as bovine) IgG (such as IgG1) heavy chain constant region are used to replace the corresponding regions of the human IgG heavy chain gene. In another embodiment, the gene encoding the one or more of the CH1-CH4 domains and/or the TM1 and TM2 domains of an ungulate-derived (such as bovine) IgG (such as IgG1) heavy chain constant region are used to replace the corresponding regions of the human IgG heavy chain gene.

The disclosure further provides transgenic ungulates comprising a HAC vector according to any embodiment or combination of embodiments of the disclosure. The transgenic ungulate comprising the HAC vector of the present invention refers to an animal into which the human artificial chromosome vector of the present invention is introduced. The transgenic ungulate having the HAC of the present invention is not particularly limited so long as the animal is a transgenic ungulate in which the human artificial chromosome fragment may be introduced into a cell thereof, and any non-human animals, for example, ungulates such as cows, horses, goats, sheep, and pigs; and the like may be used. In one aspect, the transgenic ungulate is a bovine. A transgenic ungulate having the HAC vector of the present invention may be constructed, for example, by introducing the HAC vector of the present disclosure into an oocyte of a host animal using any suitable technique, such as those described herein. The HAC vector of the present invention may, for example, be introduced into a somatic cell derived from a host ungulate by a microcell fusion method. Thereafter, the animal having the HAC vector may be constructed by transplanting a nucleus or chromatin agglomerate of the cell into an oocyte and transplanting the oocyte or an embryo to be formed from the oocyte into the uterus of a host animal to give birth. It may be confirmed by a method of Kuroiwa et al. (Kuroiwa et al., Nature Biotechnology, 18, 1086-1090, 2000 and Kuroiwa et al., Nature Biotechnology, 20, 889-894) whether an animal constructed by the above method has the human artificial chromosome vector.

The disclosure further provides transgenic ungulates comprising genes integrated into its genome encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;

wherein at least one class switch regulatory element of the genes encoding the one or more human antibody heavy chains is replaced with an ungulate-derived class switch regulatory element.

In such embodiments, the transgenic ungulate may comprise any embodiment or combination of embodiments of the nucleic acids as described herein for the HAC, but rather than being present in a HAC, they are integrated into a chromosome of the ungulate.

The disclosure further provides a method of producing a human antibody, comprising: (a) administering human thymocytes, or other target antigen of the disclosure, to the transgenic ungulate of any embodiment or combination of embodiments of the disclosure to produce and accumulate a population of human immunoglobulins specific to human thymocytes (or to T cells, B cells, and/or monocytes) in the serum or plasma of the ungulate; and optionally (b) isolating, recovering, and/or purifying the population of human immunoglobulins specific to the human thymocytes (or to T cells, B cells, and/or monocytes) from the serum or plasma of the ungulate.

The polyclonal serum or plasma, or human immunoglobulin purified from the polyclonal serum or plasma, may be used as an ATG product.

In a variation, the disclosure provides a method of recovering the protein sequence of a human antibody comprises: (i) isolating lymphocytes from the transgenic ungulate; (ii) generating a human monoclonal antibody producing hybridoma from the lymphocytes; and (iii) recovering human monoclonal antibody specific to the human thymocytes from the hybridoma. In another embodiment, the lymphocytes from the transgenic ungulate are isolated from lymph nodes of the transgenic ungulate. In a further embodiment the transgenic ungulate is hyperimmunized with the human thymocytes or other target antigen of the disclosure.

A thymocyte-specific human immunoglobulin may be produced by immunizing the transgenic ungulate having the HAC vector with human thymocytes, or other target antigen of the disclosure, to produce the thymocyte-specific human immunoglobulin in the serum or plasma of the transgenic ungulate and recovering the thymocyte-specific human immunoglobulin from the serum or plasma of the transgenic ungulate.

Examples of methods for detecting and measuring the thymocyte-specific human immunoglobulin in a composition include a binding assay by an enzyme-linked immunosorbent assay, and the like. The binding amount of a human immunoglobulin may be measured by incubating the composition comprising the human immunoglobulin with cells (e.g., thymocytes, T cells, B cells and/or monocytes, or recombinant protein antigen(s)), and then using an antibody specifically recognizing human immunoglobulin.

In a variation, the methods of the disclosure are used to generate a monoclonal antibody. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Kohler and Milstein, Nature 256:495 (1975)). An example of a preparation method for hybridomas comprises the following steps of: (1) immunizing a transgenic ungulate with thymocytes; (2) collecting antibody-producing cells from the transgenic ungulate (i.e. from lymph nodes); (3) fusing the antibody-producing cells with myeloma cells; (4) selecting hybridomas that produce a monoclonal antibody specific to thymocytes from the fused cells obtained in the above step; and optionally (5) selecting a hybridoma that produces a monoclonal antibody specific to thymocytes from the selected hybridomas.

In embodiments of the methods of producing anti-thymocyte globulin (ATG) of the disclosure, the transgenic ungulate produces human anti-thymocyte globulin (ATG). The method may comprise collecting the polyclonal serum and/or polyclonal plasma from the transgenic ungulate. In some embodiments, the ungulate is a bovine. In some embodiments, the polyclonal immunoglobulin composition comprises a population of fully human immunoglobulins, or of substantially human immunoglobulins.

Some embodiments of the methods of the disclosure, and related compositions, have the surprising advantage that the thymocyte-specific immunoglobulins are produced in high yield, in high purity, and/or as a high percentage of total immunoglobulin present in the serum or plasma of the transgenic ungulate. In some embodiments, the ungulate is a bovine.

In some embodiments of the methods and compositions of the disclosure, the polyclonal serum or polyclonal plasma comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 1-4%, 2-5%, 3-6%, 4-7%, 5-8%, 6-9%, or 7-10% fully human (or substantially human) immunoglobulin by mass of total immunoglobulin in the polyclonal serum or polyclonal plasma.

In some embodiments of the methods and compositions of the disclosure, the polyclonal immunoglobulin comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal immunoglobulin.

In some embodiments of the methods and compositions of the disclosure, the polyclonal immunoglobulin comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal immunoglobulin.

In some embodiments of the methods and compositions of the disclosure, the polyclonal immunoglobulin comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 1-4%, 2-5%, 3-6%, 4-7%, 5-8%, 6-9%, or 7-10% fully human (or substantially human) immunoglobulin by mass of total immunoglobulin in the polyclonal immunoglobulin.

In some embodiments of the methods and compositions of the disclosure, the polyclonal immunoglobulin comprises at least 5% fully human immunoglobulin by mass of total immunoglobulin in the polyclonal immunoglobulin.

In some embodiments of the methods and compositions of the disclosure, the polyclonal immunoglobulin comprises 2% to 5% fully human immunoglobulin by mass of total immunoglobulin in the polyclonal immunoglobulin.

In some embodiments, the ungulate-derived polyclonal immunoglobulin comprises “chimeric” human immunoglobulin having a human heavy chain and an ungulate kappa light chain (termed “cIgG”). In some embodiments, the polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgG as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgG as a percent of total protein concentration. In some embodiments, the polyclonal immunoglobulin comprises about 0.5% to about 1.0%, about 1.0% to about 2.0%, or about 1.0 to about 3.0% cIgG as a percent of total protein concentration.

In some embodiments, the polyclonal immunoglobulins of the disclosure are more potent in a complement-dependent cytotoxicity (CDC) assay than a reference product (e.g. Thymoglobulin or ATGAM). In some embodiments, the polyclonal immunoglobulins of the disclosure are at least about 5%, at least about 10%, at least about 25%, at least about 50%, at least about 100%, at least about 150%, or more at least about 200% potent in a complement-dependent cytotoxicity (CDC) assay than a reference product (e.g. Thymoglobulin or ATGAM).

In some embodiments, the polyclonal immunoglobulins of the disclosure generates higher toxicity towards CD8+ cells than a reference product (e.g. Thymoglobulin or ATGAM. In some embodiments, the polyclonal immunoglobulins of the disclosure are at least about 5%, at least about 10%, at least about 25%, at least about 50%, at least about 100%, at least about 150%, or at least about 200% more potent in CD8+ cell killing assay than a reference product (e.g. Thymoglobulin or ATGAM).

In some embodiments, the polyclonal immunoglobulins of the disclosure generates lower rates of CD4+ T cell apoptosis than a reference product (e.g. Thymoglobulin or ATGAM. In some embodiments, the polyclonal immunoglobulins of the disclosure are at least about 5%, at least about 10%, at least about 25%, at least about 50%, at least about 100%, at least about 150%, or at least about 200% less toxic in a CD4+ cell apoptosis assay than a reference product (e.g. Thymoglobulin or ATGAM).

In some embodiments, the polyclonal immunoglobulins of the disclosure better preserves T_{reg} to conventional T cell rations than a reference product (e.g. Thymoglobulin or ATGAM. In some embodiments, the polyclonal immunoglobulins of the disclosure are at least about 5%, at least about 10%, at least about 25%, at least about 50%, at least about 100%, at least about 150%, or at least about 200% less toxic to T_{reg} cells than a reference product (e.g. Thymoglobulin or ATGAM).

In some embodiments of the methods and compositions of the disclosure, the population of fully human immunoglobulins (or substantially human) specifically binds human thymocytes, T cells, B cells, and/or monocytes. In some embodiments, the population of fully human (or substantially human) immunoglobulins specifically binds human thymocytes.

The disclosure further provides compositions produced by immunizing a transgenic ungulate with human thymocytes, wherein the composition comprises a population of fully human or substantially human immunoglobulins and wherein the population of fully human or substantially human immunoglobulins specifically binds human thymocytes, T cells, B cells, and/or monocytes.

In some embodiments, a genome of the transgenic ungulate comprises a human immunoglobulin locus.

In some embodiments, the transgenic ungulate is immunized 3, 4, 5, or more times.

In some embodiments, the population of fully human or substantially human immunoglobulins are purified from the serum of the transgenic ungulate after immunization.

The disclosure provides methods of providing anti-thymocyte globulin (ATG) treatment to a subject in need thereof, comprising administering to the subject a polyclonal immunoglobulin according to the disclosure. In some embodiments, the method provides an effective amount of anti-thymocyte globulin (ATG) to the subject. In some embodiments, the subject suffers from type 1 diabetes. In some embodiments, the subject is an organ-transplant recipient. In some embodiments, the subject suffers from or is at risk for graft-versus-host disease. In some embodiments, the subject is a stem-cell-transplant recipient.

The disclosure provides methods of providing anti-thymocyte globulin (ATG) treatment to a subject in need thereof, comprising administering to the subject a composition produced by immunizing a transgenic ungulate with human thymocytes. In some embodiments, the method provides an effective amount of anti-thymocyte globulin (ATG) to the subject. In some embodiments, the subject suffers from type 1 diabetes. In some embodiments, the subject is an organ-transplant recipient. In some embodiments, the subject suffers from or is at risk for graft-versus-host disease. In some embodiments, the subject is a stem-cell-transplant recipient.

The disclosure provides methods of providing anti-thymocyte globulin (ATG) treatment to a subject in need thereof, comprising administering to the subject a polyclonal immunoglobulin produced according to the disclosure. In some embodiments, the method provides an effective amount of anti-thymocyte globulin (ATG) to the subject. In some embodiments, the subject suffers from type 1 diabetes. In some embodiments, the subject is an organ-transplant recipient. In some embodiments, the subject suffers from or is at risk for graft-versus-host disease. In some embodiments, the subject is a stem-cell-transplant recipient.

Illustrative methods for treatment with ATG are provided in, for example, the following references:
Voltarelli, J. C., et al. (2007) Autologous nonmyeloablative hematopoietic stem cell transplantation in newly diagnosed type 1 diabetes mellitus. JAMA. 297(14):1568-76.
Couri, C. E., et al. (2009) C-peptide levels and insulin independence following autologous nonmyeloablative hematopoietic stem cell transplantation in newly diagnosed type 1 diabetes mellitus. JAMA. 301(15):1573-9.
Haller, M. J., et al., (2015) Anti-thymocyte globulin/G-CSF treatment preserves beta cell function in patients with established type 1 diabetes. J Clin Invest. 125(1):448-55.
Haller, M. J., et al., (2018) Low-Dose Anti-Thymocyte Globulin (ATG) Preserves beta-Cell Function and Improves HbA1c in New-Onset Type 1 Diabetes. Diabetes Care. 41(9):1917-1925.

The disclosure further provides pharmaceutical compositions, comprising a population of fully human or substantially human immunoglobulins, and one or more pharmaceutically acceptable excipients. In some embodiments, the population of fully human or substantially human immunoglobulins specifically binds human thymocytes, T cells, B cells, and/or monocytes.

In some embodiments, the pharmaceutical composition comprises at least about 1 mg/mL, at least about 50 mg/mL, at least about 100 mg/mL, or at least about 1,000 mg/mL of fully human or substantially human immunoglobulin. In some embodiments, the pharmaceutical composition comprises at least about 100 μg/mL, at least about 250 μg/mL, at least about 500 μg/mL, at least about 750 μg/mL, or at least about 1,000 μg/mL of fully human or substantially human immunoglobulin.

In some embodiments, the fully human or substantially human immunoglobulin is produced in an ungulate. In some embodiments, the ungulate is a bovine.

In some embodiments, the pharmaceutical composition comprises at least 5% fully human immunoglobulin by mass of total immunoglobulin in the pharmaceutical composition.

In some embodiments, the pharmaceutical composition comprises 2% to 5% fully human immunoglobulin by mass of total immunoglobulin in the pharmaceutical composition.

### EXAMPLES

The following specific examples are to be construed as merely illustrative, and not limitative of the remainder of the disclosure in any way whatsoever.

### Example 1

### Production of Human Polyclonal ATG in Transchromosomic Bovine (TcB) System

We report development of a novel human polyclonal ATG product (termed herein the “TcB product”) that overcomes known limitations of animal ATGs. We utilized the diversitAb™ platform technology, a transchromosomic bovine (TcB) system, in which cows with a bovine Ig locus replaced by a human artificial chromosome express fully human polyclonal antibodies.

A TcB subject was immunized with human thymocytes and adjuvant at 3-5 week intervals. Hyperimmune plasma was collected after the 3rd-5th vaccinations (V3-V5). Immunization study design is summarized in Table 1. The amount of hyperimmune plasma collected from the subject animals at days 7, 11, and 14 after vaccination 5 (V5) collected was 2.1% of plasma by weight of animal (BW)

TABLE 1

| Vaccination | | |
|---|---|---|
| (3-6 week interval) | Vaccine Formulation | Bleeds (D = day) |
| V1 | 2 × 10⁹ fresh thymocytes + | D0, D4, D20 |
| | adjuvant | |
| V2 | 2 × 10⁹ fresh thymocytes + | D11, D14, D21 |
| | adjuvant | |
| V3 | 2 × 10⁹ fresh thymocytes + | D7, D11, D14 |
| | adjuvant | |
| V4 | 5.5 × 10⁹ fresh thymocytes + | D7, D11, D14 |
| | adjuvant | |
| V5 | 4.12 × 10⁹ fresh thymocytes + | D7, D11, D14 |
| | adjuvant | |

Complement-Dependent Cytotoxicity

Complement-dependent cytotoxicity (CDC) was comparable to ATGAM and Thymoglobulin with an increase in SAB-ATG potency from V3/V4 to V5. Concentration of immunoglobulin in each sample was measured by NanoDrop™ spectrophotometer instrument that measures total protein at a wavelength of 260 nm.

The CDC assay is a cytometry-based assay in which sera, plasma, in-process, or purified antibody products are incubated with human PBMC followed by incubation with rabbit complement. Antibodies specific for human lymphocytes will bind to the cells, and complement will then in turn bind to both the immunoglobulin and the cell. Complement is a cascade of proteins that, upon binding to cells, eventually leads to cell lysis. Cell death is measured using cellular viability dyes, such as ViaCount Reagent®. The proportion of viable cells is calculated when the sample is read on the flow cytometer. By plotting percent cell viability against antibody concentration, the LT₂₅ value can be calculated. This value indicates the amount of antibody required to kill 25% of the cells. A lower value translates to higher ATG potency or activity. This value can then be standardized using the CF₂₅ if desired.

Results for a CDC assay using rabbit complement are shown in Table 2. With rabbit complement the TcB product had CDC potency similar to rabbit-derived Thymoglobulin®.

TABLE 2

| | Concentration | | LT₂₅ | CF₂₅ |
|---|---|---|---|---|
| Sample ID | (mg/mL) | R² | (μg/mL) | (μg/mL) |
| Thymoglobutin | 5.0 | 1.000 | 2.423 | 1.70 |
| ATGAM | 50.0 | 1.000 | 8.174 | 5.72 |
| 2322 V3D11 | 16.39 | 1.000 | 2.586 | 1.81 |
| 2322 V4D11 | 20.11 | 1.000 | 2.474 | 1.73 |
| 2322 V5D7 | 18.58 | 1.000 | 5.590 | 3.91 |
| 2322 V5D11 | 17.67 | 0.999 | 3.776 | 2.64 |

LT₂₅ means IgG concentration at which 25% of human PBMCs are lysed in the presence of rabbit complement.
CF₂₅ means 25^{th} percentile cytotoxicity factor.
It is a standardization of LT₂₅ to account for assay variation, calculated as (Sample LT₂₅ / Reference LT₂₅) × Reference LT₂₅.

In a further CDC assay, hyperimmune plasma after vaccination 3 (V3) or vaccination 5 (V5) were absorbed onto human red blood cells (RBCs). CDC potency was determined using a rabbit complement. Results for a CDC assay using rabbit complement are shown in Table 3. Lower values indicate greater potency.

TABLE 3

| | LT₅₀ (μg/mL) |
|---|---|
| Thymoglobulin | 6.18 |
| SAB-ATG V3 | 5.57 |
| Non RBC Adsorped | |
| SAB-ATG V3 | 7.31 |
| RBC Adsorped | |
| SAB-ATG V5 | 6.08 |
| Non RBC Adsorped | |
| SAB-ATG V5 | 6.99 |
| RBC Adsorped | |

LT50 means IgG concentration at which 50% of human PBMCs are lysed in the presence of rabbit complement.
LT50 value is the average from two day assays.

With human complement, the TcB product had CDC potency similar to rabbit-derived Thymoglobulin® (data not shown).

Biochemical Characterization

To determine the concentration of human immunoglobulin, and confirm the absence of bovine immunoglobulin in the plasma samples, a portion of the product was absorbed onto human red blood cells (RBCs

Size exclusion chromatography and SDS-PAGE confirmed that the same contained predominantly well folded and non-aggregated paired heavy and light chain IgG molecules. Only about 5.6% (=(62.1−58.6)/62.1) of total protein was adsorbed to RBCs, demonstrating that only a minor fraction of the immunoglobulin cross-reacts with RBCs even without purification by adsorption to RBCs.

Binding to Human PBMCs

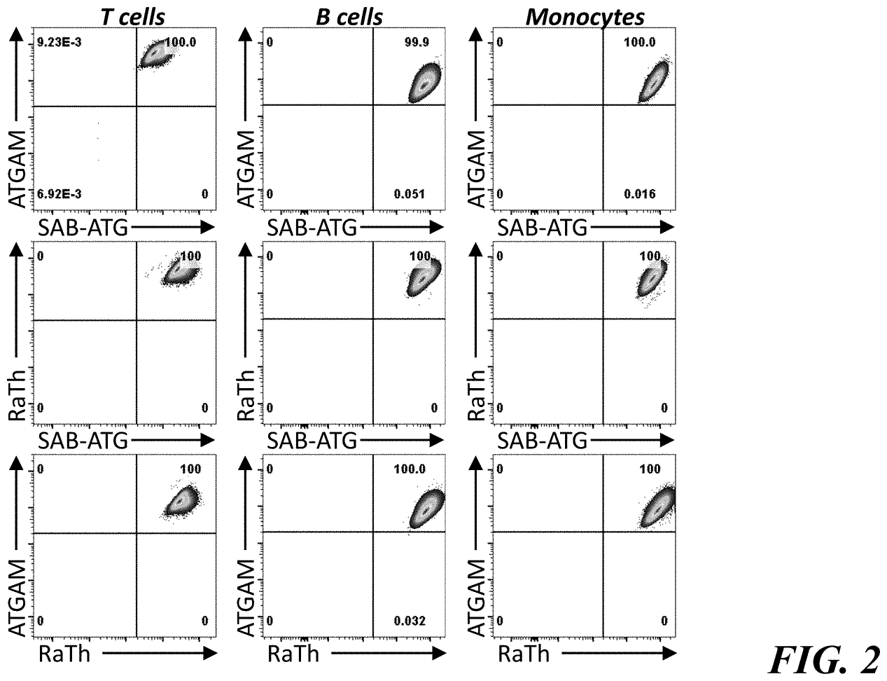

Flow cytometry assessment (FIG. 2) of binding to human PBMCs: pan-T cells, CD4+ and CD8+ conventional T cells, Tregs, NK T cells, B cells, and neutrophils revealed that the TcB product has specificity for T cells, B cells, and/or monocytes that is identical to horse-derived (ATGAM) and rabbit-derived (Thymoglobulin) ATG. No single-positive cells and highly similar mean fluorescence intensities (MFIs) were observed. The rare (˜0.4%) RBCs stained by the TcB product were also stained by ATGAM and Thymoglobulin, confirming that the TcB product has no unique RBC specificity that might contraindicate human use.

T Cell Killing

ATG products achieve their medical effect in part by killing T cells. Therefore, in vitro T cell killing is a commonly used surrogate for in vivo efficacy of an ATG product.

In non-activating conditions, the TcB product, surprisingly, possessed significantly higher toxicity towards CD8+ cells compared to Thymoglobulin, while ATGAM was least potent. The TcB-product treatment, again surprisingly, induced lower rates of CD4+ T cell apoptosis, preserving more CD4+ cells than Thymoglobulin, with increased preservation of Tregs compared to conventional T cells. Results with non-activated T cells are shown in Table 4 (percentage indicated cellular population, +/− standard deviation).

TABLE 4

| | | Live T cells | | Live CD4+ | Live CD8+ | | |
|---|---|---|---|---|---|---|---|
| | | (relative to | Apoptotic | T cells | T cells | Apoptotic | Apoptotic |
| | Conc. | Control IgG | lymphocytes | (relative to | (relative to | CD4+ T cells | CD8+ T cells |
| Treatment | (mg/ml) | treatment) | (AnnV+, PI−) | control IgG) | Control igG) | (AnnV+, PI−) | (AnnV+, PI−) |
| Control human igG | 5 | 100% (±0.7) | 3.2% (±1.3) | 100% (±31) | 100% (±32) | 0.9% (±0.4) | 5.8% (±0.1) |
| ATGAM | | 95.7% (±1.9) | 4.8% (±1) | 72.6% (±19.6) | 74.9% (±19.5) | 3.2% (±0.7) | 7.3% (±0.1) |
| Thymoglobulin | | 85.8% (±1.5) | 5.8% (±1.3) | 69.2% (±13.6) | 69% (±12.6) | 3.4% (±1) | 10.6% (±2.3) |
| SAB-ATG | | 85.4% (±6.8) | 7.1% (±0.7) | 97.3% (±31) | 94.5% (±27) | 3.5% (±0.3) | 14.5% (±0.7) |
| Control human IgG | 10 | 95.9% (±1.3) | 3.2% (±1) | 87.7% (±24) | 87.6% (±24) | 0.9% (±0.4) | 5.6% (±0.1) |
| ATGAM | | 92.3% (±0.4) | 7% (±1.2) | 72.2% (±12.6) | 74.2% (±12.4) | 5.1% (±0.7) | 8.8% (±0.1) |
| Thymoglobulin | | 78.1% (±4) | 17.8% (±2.2) | 65.6% (±23.5) | 72.6% (±25.4) | 18.8% (±2.5) | 15.0% (±2.3) |
| SAB-ATG | | 76.9% (±5.8) | 11.1% (±0.2) | 82% (±19.8) | 76.5% (±15.4) | 6.9% (±0.2) | 16% (±0.9) |
| Control human IgG | 30 | 95.6% (±4.6) | 3.1% (±0.5) | 83.3% (±1.3) | 84.2% (±2.4) | 0.7% (±0.2) | 5.6% (±0.3) |
| ATGAM | | 82.7% (±2.1) | 22.1% (±0.1) | 57.9% (±1.1) | 59.2% (±2.2) | 21.8% (±0.7) | 23.6% (±0.3) |
| Thymoglobulin | | 57% (±7.2) | 39.4% (±3.1) | 24.3% (±3.4) | 42.5% (±3.4) | 50.8% (±3.3) | 37.9% (±2) |
| SAB-ATG | | 74.6% (±4) | 30.2% (±0.6) | 48.3% (±11.2) | 23.4% (±5.7) | 23% (±1.1) | 46.3% (±1.8) |
| Control human IgG | 100 | 93.5% (±4) | 2.8% (±0.8) | 77.2% (±0.7) | 73.9% (±2.1) | 0.6% (±0.1) | 5.1% (±0.4) |
| ATGAM | | 30.4% (±1) | 62.1% (±2.3) | 3.5% (±0.2) | 18.8% (±1.5) | 78.7% (±0.6) | 72% (±0.1) |
| Thymoglobulin | | 25.8% (±5.9) | 68.4% (±6.2) | 1.3% (±0.04) | 15.4% (±0.1) | 74.8% (±2.3) | 89.1% (±1.6) |
| SAB-ATG | | 69.3% (±2.6) | 36% (±2) | 30.1% (±4.4) | 10.5% (±0.05) | 31.1% (±0.6) | 99.8% (±0.6) |

After cells activation, the TcB product was cytotoxic to both CD8+ and CD4+ cells (more so to CD4+ cells). Surprisingly, the TcB product had greater potency the other ATGs. Unexpectedly, apoptotic CD8+ and CD4+ cells were fewer at higher concentrations of TcB product than the other ATGs, suggesting that the TcB product-mediated cytotoxicity is more rapid and involves additional biochemical pathways. Results with PHA-activated T cells are shown in Table 5 (percentage indicated cellular population, +/− standard deviation).

TABLE 5

| | | Live T cells | | Live CD4+ | Live CD8+ | | |
|---|---|---|---|---|---|---|---|
| | | (relative to | Apoptotic | T cells | T cells | Apoptotic | Apoptotic |
| | Conc. | Control IgG | lymphocytes | (relative to | (relative to | CD4+ T cells | CD8+ T cells |
| Treatment | (mg/ml) | treatment) | (AnnV+, PI−) | control IgG) | Control igG) | (AnnV+, PI−) | (AnnV+, PI−) |
| Control human igG | 5 | 100% (±2.8) | 6.4% (±3.4) | 100% (±10) | 100% (±20.2) | 5.1% (±2.2) | 12.7% (±10.3) |
| ATGAM | | 75% (±5.2) | 14.8% (±3.6) | 37.7% (±11.3) | 32% (±10) | 16.3% (±5.9) | 28.7% (±3.9) |
| Thymoglobulin | | 87.1% (±4.4) | 11% (±0.5) | 66.7% (±8.3) | 59.8% (±8.8) | 10.4% (±0.4) | 22.4% (±1.4) |
| SAB-ATG | | 76.8% (±2.2) | 16.3% (±0.8) | 55.6% (±0.02) | 47.4% (±3) | 19.3% (±0.4) | 30.8% (±0.3) |
| Control human IgG | 10 | 90.1% (±2) | 10.1% (±2.8) | 78.6% (±18.5) | 72.5% (±17.8) | 7.7% (±2.8) | 21.8% (±3.9) |
| ATGAM | | 79.5% (±1.3) | 15.3% (±3.6) | 34% (±11.7) | 29.1% (±13.3) | 17.7% (±1.8) | 27.3% (±6.6) |
| Thymoglobulin | | 81.2% (±0.7) | 14.2% (±0.6) | 61% (±15.2) | 53.1% (±15.2) | 17.3% (±0.1) | 24.8% (±2.1) |
| SAB-ATG | | 67% (±3.2) | 17.7% (±2.7) | 38.4% (±1.7) | 30.7% (±0.5) | 24.1% (±1.1) | 32.8% (±2.4) |
| Control human IgG | 30 | 91.2% (±3.8) | 9.4% (±1.7) | 96.2% (±19.8) | 89.1% (±21.4) | 7.3% (±1.9) | 20.8% (±2.2) |
| ATGAM | | 68.1% (±7.1) | 21.3% (±6.2) | 20% (±0.4) | 17% (±2.7) | 33% (±6.9) | 32.6% (±6.7) |
| Thymoglobulin | | 57.7% (±4.1) | 24.1% (±1.4) | 30.3% (±2.7) | 30.9% (±3.3) | 44% (±1.9) | 32.9% (±0.3) |
| SAB-ATG | | 54.4% (±1.2) | 13% (±1.6) | 18.7% (±6) | 12.1% (±2.8) | 25.7% (±1.5) | 23.7% (±4.9) |
| Control human IgG | 100 | 90.3% (±5.8) | 9.5% (±1) | 89.6% (±19.2) | 80.8% (±19.2) | 7.5% (±1.2) | 21.3% (±1) |
| ATGAM | | 55.5% (±5.6) | 33.8% (±7.5) | 26.3% (±7.1) | 20.6% (±3.3) | 70.2% (±4.9) | 61.6% (±4.6) |
| Thymoglobulin | | 53.8% (±8.6) | 22.6% (±0.2) | 19.1% (±0.3) | 21.9% (±1) | 54.9% (±2.1) | 32.8% (±0.4) |
| SAB-ATG | | 48.5% (±1.1) | 14.9% (±2.8) | 5.3% (±0.3) | 1.6% (±0.4) | 44.8% (±4.8) | 26% (±4.2) |

In summary, SAB-ATG displays binding and in vitro cytotoxicity similar to or superior to commercial ATG products.

T Cell Survival

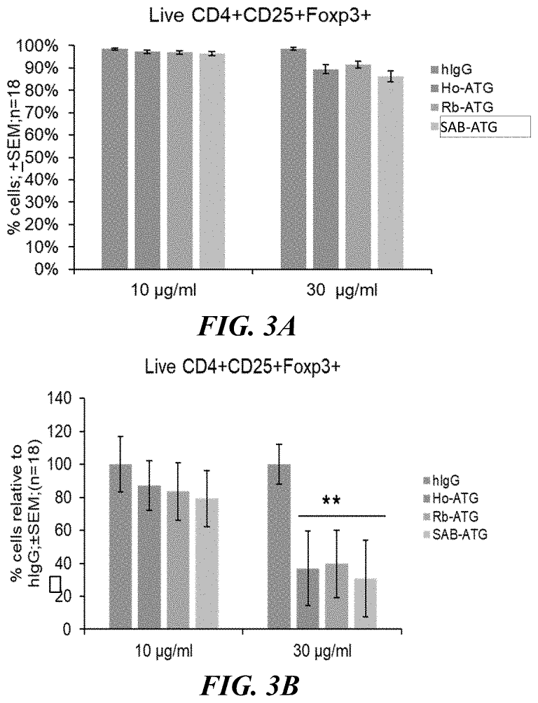

The effect of TcB product on regulatory T (Treg) cell survival was compared with Thymoglobulin® and ATGAM®. FIGS. 3A-3B show levels of regulatory T (Treg) cells treated with horse (Ho-ATG), rabbit (Rb-ATG), or TcB (SAB-ATG) products. TcB product preserved Treg cells at the level similar to Thymoglobulin®.

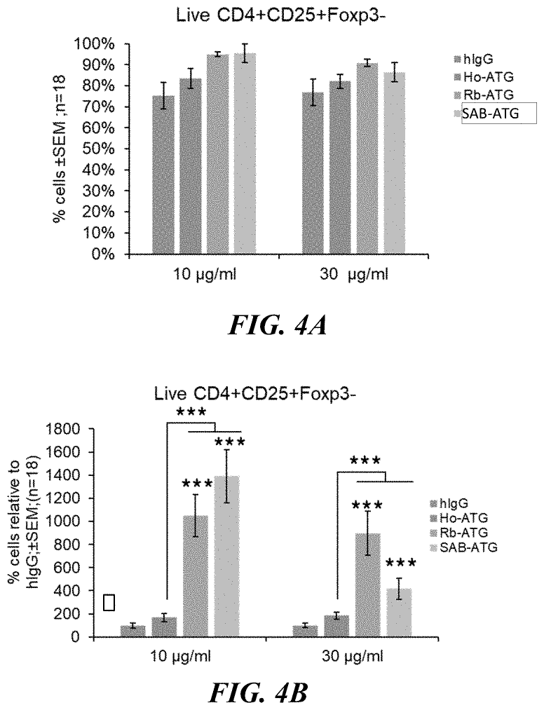

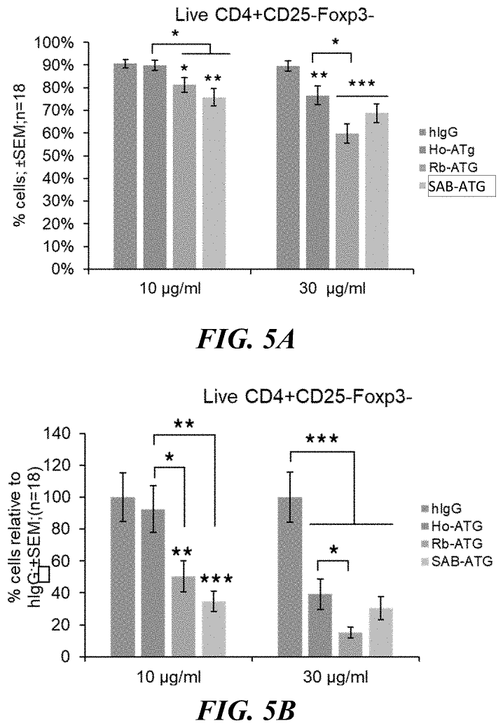

Similar assays were performed on conventional T (Tconv) cells. Results for activated and naïve Tconv cells are shown in FIGS. 4A-4B and FIGS. 5A-5B, respectively. TcB product induced the activation of T cells at the level similar to Thymoglobulin®. TcB product reduced naïve T cells at level similar to Thymoglobulin®.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An ungulate-derived polyclonal immunoglobulin composition, comprising a population of fully human or substantially human immunoglobulins,
wherein the population of fully human or substantially human immunoglobulins specifically binds human thymocytes, T cells, B cells, and/or monocytes.

2. The composition of claim 1, wherein the composition is at least about as potent in a CDC assay as a reference product.

3. The composition of claim 1, wherein the composition is at least about 10% more potent in a CDC assay than a reference product.

4. The composition of claim 1, wherein the composition is at least about 10% more potent in a CD8+ cell killing assay than a reference product.

5. The composition of claim 1, wherein the composition is at least about 10% more less in a CD4+ cell apotosis assay than a reference product.

6. The composition of any one or claim 2, wherein the reference product is a rabbit-derived ATG.

7. The composition of claim 2, wherein the reference product is a horse-derived ATG.

8. The composition of claim 1, wherein the composition comprises at least 2% fully human or substantially human immunoglobulin by mass of total immunoglobulin in the composition.

9. The composition of claim 1, wherein the ungulate is a bovine.

10. A composition, produced by immunizing a transgenic ungulate with human thymocytes, wherein the composition comprises a population of fully human or substantially human immunoglobulins, and
wherein the population of fully human or substantially human immunoglobulins specifically binds human thymocytes, T cells, B cells, and/or monocytes.

11-21. (canceled)

22. A method of producing anti-thymocyte globulin (ATG), comprising administering human thymocytes to a transgenic ungulate, wherein the transgenic ungulate comprises a genome comprising a human immunoglobulin locus or an artificial chromosome comprising a human immunoglobulin locus, wherein the transgenic ungulate produces human anti-thymocyte globulin (ATG).

23. The method of claim 22, comprising administering the thymocytes 3, 4, 5, or more times.

24. The method of claim 22, comprising collecting serum or plasma from the transgenic ungulate.

25. The method of claim 22, wherein the serum or plasma comprises a population of fully human immunoglobulins.

26-31. (canceled)

32. A method of providing anti-thymocyte globulin (ATG) treatment to a subject in need thereof, comprising administering to the subject the composition of claim 1,
wherein the method provides an effective amount of anti-thymocyte globulin (ATG) to the subject.

33. The method of claim 32, wherein the subject suffers from type 1 diabetes.

34. The method of claim 32, wherein the subject is an organ-transplant recipient.

35. The method of claim 32, wherein the subject suffers from or is at risk for graft-versus-host disease.

36. The method of claim 32, wherein the subject is a stem-cell-transplant recipient.

37. A pharmaceutical composition comprising a population of fully human or substantially human immunoglobulins, and one or more pharmaceutically acceptable excipients,
wherein the population of fully human or substantially human immunoglobulins specifically binds human thymocytes, T cells, B cells, and/or monocytes.

38-47. (canceled)
